(19) United States
(12) Patent Application Publication
(10) Pub. No.: US 2026/0355081 A1
(43) Pub. Date: Nov. 12, 2026

(54) SOLID COMPOSITION COMPRISING A CATIONIC SURFACTANT, A STARCH, AN AMPHOTERIC SURFACTANT AND A FATTY SUBSTANCE

(71) Applicant: L'Oreal, Paris (FR)

(72) Inventors: Andreia Teixeira, Saint Ouen (FR); Valentine Bayle, Saint Ouen (FR)

(73) Assignee: L'Oreal, Paris (FR)

(21) Appl. No.: 19/120,166

(22) PCT Filed: Oct. 26, 2023
(86) PCT No.: PCT/EP2023/079907

§ 371 Date: Apr. 10, 2025

(30) Foreign Application Priority Data
Oct. 26, 2022 (FR) FR2211145

(51) Int. Cl. A61K 8/41; A45D 19/00; A45D 40/24; A61K 8/44; A61K 8/73; A61K 8/92; A61Q 5/12

(52) U.S. Cl. CPC A61K 8/416; A45D 19/005; A45D 40/24; A61K 8/442; A61K 8/732; A61K 8/922; A61Q 5/12; A61K 2800/87

(57) ABSTRACT

The present invention relates to a solid composition intended in particular for caring for keratin fibres, notably human keratin fibres such as the hair, and which comprises a cationic surfactant, a starch, one or more amphoteric surfactants and one or more fatty substances. The invention also relates to a packaging article containing said solid composition, and also to cosmetic processes for treating keratin fibres, in particular human keratin fibres such as the hair, using said solid composition or said packaging article. The invention also relates to the use of said solid composition or of said packaging article for caring for keratin fibres, in particular human keratin fibres such as the hair.

The present invention relates to a solid composition intended in particular for the cosmetic treatment, and more particularly the care, of keratin fibres, notably human keratin fibres such as the hair, and which comprises at least one cationic surfactant, at least one starch, at least one amphoteric surfactant and at least one fatty substance.

The invention also relates to a packaging article containing said solid composition, and also to cosmetic processes for treating keratin fibres, in particular human keratin fibres such as the hair, using said solid composition or said packaging article.

The invention also relates to the use of said solid composition or of said packaging article for the cosmetic treatment, preferably the care, of keratin fibres, in particular human keratin fibres such as the hair.

In the field of hair hygiene, products for caring for (or conditioning) keratin fibres are generally intended to condition said fibres in order to give them good cosmetic properties. Conventional products, such as conditioners, are usually in more or less thickened liquid form. However, on account of their liquid texture, these products may have various drawbacks, and may notably prove to be difficult to measure out.

The reason for this is that the more liquid they are, the greater their tendency to escape between the fingers, making them difficult to measure out and leading to waste. These products may also escape from their packaging, which is a source of inconvenience to the consumer when these products come into contact with clothing or objects, for example when travelling.

In order to modify the texture of these products, and notably to make it more compact, thickeners are generally used. However, the addition of these compounds usually comes at the expense of the cosmetic effects of the compositions. The use of these thicker compositions moreover necessitates a large amount of rinsing water in order to remove the surplus of product on the fibres. Now, in many countries where access to water is restricted, the rinsing time and consequently the amount of water required to properly rinse off the product are key indicators of the working qualities of a composition.

In order to overcome some of these problems, novel solid cosmetic formulations, notably conditioners in the form of solid granules or powder, have been developed. Such formulations are described, for example, in US 2021/007960. However, these novel formulations are not always entirely satisfactory. Those which are in loose powder form may, indeed, pose problems of volatility, uptake and/or measuring out. Those which are in the form of agglomerates, for instance granules, may have a tendency to disintegrate or break down with difficulty in the presence of water, having a negative impact on their use and their spreading on keratin fibres, and not always permitting satisfactory care to be obtained. They may also be difficult to remove on rinsing and may occasionally even leave residues on the fibres, which the consumer finds unpleasant.

Conditioners in powder or particle form may lose fluidity during storage due to the agglutination of the individual solid particles with each other, which may have a negative impact on the working qualities.

These formulations may also not be entirely satisfactory in terms of cosmetic performance qualities, notably in terms of suppleness, feel, softness, disentangling, smoothness and sheen.

Thus, there is a real need to provide a composition in solid form which has an improved environmental profile, i.e. which requires little water throughout its use. The composition must not only be easy to take up and break down easily, but must also rinse out quickly without leaving residues on the keratin fibres. As regards solid compositions notably in powder or particle form, they must not agglutinate together on storage, so as not to deteriorate the working qualities.

The composition must also afford good cosmetic properties, notably in terms of suppleness, feel, softness, coating, sheen and disentangling.

It has now been found that a solid composition comprising at least one cationic surfactant, at least one starch, at least one amphoteric surfactant and at least one fatty substance makes it possible to achieve the objectives presented above, and notably to propose a composition in solid form which combines good conditioning power, without, however, requiring large amounts of water.

One subject of the present invention is thus a solid composition comprising:
i) one or more cationic surfactants,
ii) one or more starches,
iii) one or more amphoteric surfactants,
iv) one or more fatty substances.

Said solid composition may more particularly be a cosmetic composition, notably a hair composition, and advantageously, a hair conditioning composition.

The particular combination of the compounds of the invention makes it possible to obtain a solid composition that is easy to take up, to handle and to measure out. Specifically, the composition thus obtained has a cohesion or granulation such that the uptake and measuring-out properties are improved, while at the same time avoiding undesired agglomerates which have a negative impact on the working qualities. The composition can then be packaged in single-dose form, which is a form that is particularly advantageous, for example, when travelling or performing a sporting activity (lightened bags, limited risks of leakage, reduced waste).

In addition, this composition breaks down rapidly on contact with water and makes it possible readily and quickly to obtain easy spreading and uniform distribution over the keratin fibres comparable to those of a conventional liquid conditioner composition.

The composition according to the invention also makes it possible to obtain good coating of the keratin fibres, with a uniform and gliding finish.

Moreover, the composition of the invention rinses out rapidly without leaving unpleasant residues on the fibres and gives them a natural, clean feel after rinsing. Fibres treated with the composition of the invention have good cosmetic properties, notably in terms of softness, suppleness and feel. They also have good strand separation and are thus easier to disentangle.

A subject of the present invention is also a process for the cosmetic treatment, notably for the care, of keratin fibres, in particular human keratin fibres such as the hair, comprising the application to said keratin fibres of a solid composition as defined below, the solid composition being applied directly to said keratin fibres or after having been moistened beforehand with water.

The present invention also relates to the use of a solid composition as defined below for the cosmetic treatment, preferably the care, of keratin fibres, in particular human keratin fibres such as the hair.

The present invention also relates to a packaging article comprising:
an envelope defining at least one cavity, the envelope comprising one or more water-soluble and/or liposoluble compounds;
a solid composition comprising: i) one or more cationic surfactants, ii) one or more starches, iii) one or more amphoteric surfactants, and iv) one or more fatty substances;
it being understood that the solid composition is in one of the cavities defined by the envelope.

This packaging article notably solves the problems of measuring out of the solid composition. It also facilitates its storage and transportation. In particular, the packaging article of the invention affords better protection of the composition against moisture.

The packaging article may also make it possible to obtain a final keratin fibre care composition that is more thickened in the hand, which may be in cream form, without lumps, without leaving residues on the hair after rinsing.

The packaging article may thus improve, notably facilitate, the distribution of the composition over keratin fibres.

The packaging article may make it possible to better control the dose of composition employed on keratin fibres, thus reducing the risks of waste.

The packaging article also makes it possible to minimize the risks of breaking down of the composition.

The invention also relates to the use of the above packaging article for the cosmetic treatment, preferably for the care, of keratin fibres, in particular human keratin fibres such as the hair.

The invention also relates to a process for the cosmetic treatment, notably for the care, of keratin fibres, in particular human keratin fibres such as the hair, comprising a step of using at least one packaging article as defined above.

Preferably, said cosmetic treatment process comprises the following steps:
a) mixing the packaging article in a composition that is capable of dissolving, totally or partially, the envelope of said packaging article,
b) applying the composition obtained in step a) to the keratin fibres,
c) optionally leaving to stand,
d) rinsing said keratin fibres,
e) optionally drying said keratin fibres.

Other subjects, characteristics, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

Preferably, the solid composition according to the invention (final composition, after drying) has a water activity of less than 0.75, better still of less than 0.72, even better still of less than 0.70. The water activity, Aw, represents the proportion of bonded water relative to free water which is conducive to the growth of microorganisms. The value varies between 0 and 1. The activity is 0 for a completely dry product and it is 1 for pure water.

The solid composition according to the invention may comprise water added during its preparation and/or water that may originate from the starting materials used during the preparation of said composition.

The solid composition according to the invention may be in powder, paste, particle (for example spherical particles such as small beads or granules), compressed tablet, stick or cake form. Preferably, the composition according to the invention is in the form of a powder or of particles.

The term “powder” means a composition in pulverulent form, which is preferably essentially free of dust (or fine particles). In other words, the particle size distribution of the particles is such that the weight content of particles which have a size of less than or equal to 50 μm (content of fines), preferably less than or equal to 45 μm (content of fines) is advantageously less than or equal to 5% by weight, preferably less than 3% by weight and more particularly less than 1% by weight, relative to the total weight of particles (particle size evaluated using a Retsch AS 200 Digit particle size analyser; oscillation height: 1.25 mm/screening time: 5 minutes).

The term “paste” means a composition having a viscosity of greater than 0.5 Pa·s (5 poises) and preferably greater than 1 Pa·s (10 poises), measured at 25 °C and at a shear rate of 1 s⁻¹; this viscosity possibly being determined using a cone-plate rheometer.

The term “particles” means small fractionated objects formed from solid particles that are aggregated together, of variable shapes and sizes. They may be in regular or irregular form. They may in particular be in spherical form (such as granules, granulates or beads) or in square, rectangular or elongated form such as sticks. Spherical particles are most particularly preferred.

Advantageously, the solid composition is in powder form.

Advantageously, the size of the powders or particles is, in its largest dimension, between 30 μm and 5 mm, more particularly between 45 μm and 2 mm, better still between 50 μm and 1 mm and even better still between 60 and 700 μm.

Advantageously, the solid composition is in powder form, the size of the powders being, in its largest dimension, between 30 μm and 5 mm, and more particularly between 45 μm and 2 mm, better still between 50 μm and 1 mm, even better still between 60 and 700 μm.

When the solid composition according to the invention is not in powder or particle form, it advantageously has a penetration force at 25 °C and under 1.013×105 Pa (1 atm) of greater than or equal to 200 g, preferably greater than or equal to 300 g, more preferentially greater than or equal to 400 g and better still greater than or equal to 500 g. The penetration force is determined by penetrometry. The texture analysis measurements are performed at 25 °C using a Stable Micro Systems TA.XT Plus texturometer. The penetrometry experiments are performed with a metal rod equipped with a screwed end piece, said end piece being a P/2N needle of 2 mm for the top part, connected to the measuring head. The piston penetrates into the sample at a constant speed of 1 mm/s, to a depth of 5 mm. The force exerted on the piston is recorded and the mean value of the force is calculated.

The solid composition according to the invention may be in the form of a compressed solid composition, notably compressed using a manual or mechanical press. Preferably, the hardness of the compressed solid composition is between 10 and 300 N, better still between 15 and 200 N and even better still between 15 and 100 N.

The density of the solid composition according to the present invention is preferably between 0.1 and 1, more preferentially between 0.2 and 0.8 and better still between 0.3 and 0.7. It is measured as follows:
a given amount (mass, m) of powder is placed in a measuring cylinder. The measuring cylinder is then automatically tapped 2500 times. The volume (v) thus obtained is read on the measuring cylinder and the density (d) is then determined according to the formula d=m/v.

### Cationic Surfactants(s) i)

The solid composition according to the present invention comprises one or more cationic surfactants i).

The term “cationic surfactant” means a surfactant that is positively charged when it is contained in the compositions according to the invention. This surfactant may bear one or more positive permanent charges or may contain one or more cationizable functions within the compositions according to the invention.

The cationic surfactants are advantageously chosen from optionally polyoxyalkylenated primary, secondary or tertiary fatty amine salts, quaternary ammonium salts, and mixtures thereof.

As quaternary ammonium salts, mention may notably be made of:
the quaternary ammonium salts of formula (Ia):

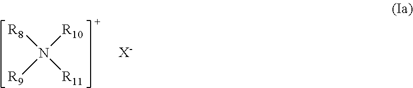

in which:
the groups R₈ to R₁₁, which may be identical or different, represent a linear or branched aliphatic group including from 1 to 30 carbon atoms, or an aromatic group such as aryl or alkylaryl, at least one of the groups R₈ to R₁₁ including from 8 to 30 and preferably from 12 to 24 carbon atoms, it being possible for the aliphatic groups to include heteroatoms notably such as oxygen, nitrogen, sulfur and halogens; and
X⁻ is an anion notably chosen from the group of halides, phosphates, acetates, lactates, (C₁-C₄)alkyl sulfates, (C₁-C₄)alkylsulfonates or (C₁-C₄)alkylarylsulfonates.

The aliphatic groups R₈ to R₁₁ may be chosen from C₁-C₃₀ alkyl, C₁-C₃₀ alkoxy, (C₂-C₆) polyoxyalkylene, C₁-C₃₀ alkylamide, (C₁₂-C₂₂)alkylamido(C₂-C₆)alkyl, (C₁₂-C₂₂)alkyl acetate, and C₁-C₃₀ hydroxyalkyl groups.

Mention may notably be made of tetraalkylammonium halides, notably chlorides, such as dialkyldimethylammonium or alkyltrimethylammonium chlorides in which the alkyl group includes from 12 to 22 carbon atoms, in particular behenyltrimethylammonium chloride, distearyldimethylammonium chloride, cetyltrimethylammonium chloride and benzyldimethylstearylammonium chloride.

Mention may also be made of palmitylamidopropyltrimethylammonium or stearamidopropyldimethyl-(myristyl acetate)-ammonium halides, and notably chlorides; notably the product sold under the name Ceraphyl® 70 by the company Van Dyk.
the quaternary ammonium salts of imidazoline of formula (Ha):

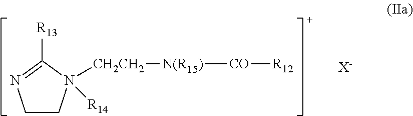

in which:
R12 represents an alkenyl or alkyl group including from 8 to 30 carbon atoms, for example tallow fatty acid derivatives;
R13 represents a hydrogen atom, a C₁-C₄ alkyl group or an alkenyl or alkyl group including from 8 to 30 carbon atoms;
R14 represents a C₁-C₄ alkyl group;
R15 represents a hydrogen atom or a C₁-C₄ alkyl group;
X⁻ is an anion notably chosen from the group of halides, phosphates, acetates, lactates, (C₁-C₄)alkyl sulfates, (C₁-C₄)alkylsulfonates or (C₁-C₄)alkylarylsulfonates.

Preferably, R12 and R13 denote a mixture of alkenyl or alkyl groups including from 12 to 21 carbon atoms, for example tallow fatty acid derivatives, R14 denotes a methyl group and R15 denotes a hydrogen atom. Such a product is sold, for example, under the name Rewoquat® W75 or W90 by the company Evonik.
the quaternary di- or triammonium salts of formula (IIIa):

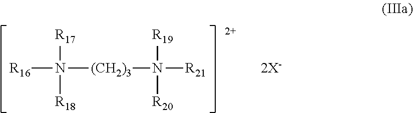

in which:
R16 denotes an alkyl group including from 16 to 30 carbon atoms, which is optionally hydroxylated and/or optionally interrupted with one or more oxygen atoms,
R17 denotes hydrogen, an alkyl group including from 1 to 4 carbon atoms or a group —(CH₂)₃—N⁺(R₁₆ₐ)(R₁₇ₐ)(R₁₈ₐ), R₁₆ₐ, R₁₇ₐ and R₁₈ₐ, which may be identical or different, denoting hydrogen or an alkyl group including from 1 to 4 carbon atoms,
R18, R19, R20 and R21, which may be identical or different, denote hydrogen or an alkyl group including from 1 to 4 carbon atoms, and
X⁻ is an anion, chosen notably from the group of halides, acetates, phosphates, nitrates, (C1-C4)alkyl sulfates, (C1-C4)alkylsulfonates and (C1-C4)alkylarylsulfonates, in particular methyl sulfate and ethyl sulfate.

Such compounds are, for example, Finquat CT-P (Quaternium 89) and Finquat CT (Quaternium 75), sold by the company Finetex;
quaternary ammonium salts containing one or more ester functions, of formula (IVa) below:

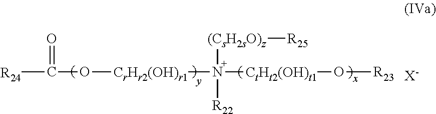

in which:
R22 is chosen from C₁-C₆ alkyl and C₁-C₆ hydroxyalkyl or dihydroxyalkyl groups;
R23 is chosen from the group R26-C(═O)—, linear or branched, saturated or unsaturated C1-C22 hydrocarbon-based groups R27, and a hydrogen atom;
R25 is chosen from the group R28-C(═O)—, linear or branched, saturated or unsaturated C₁-C₆ hydrocarbon-based groups R29, and a hydrogen atom;
R24, R26 and R28, which may be identical or different, are chosen from linear or branched, saturated or unsaturated C7-C21 hydrocarbon-based groups;
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10,
X— is an anion,
it being understood that r2+r1=2r and t1+t2=2t, and that the sum x+y+z ranges from 1 to 15,
with the proviso that when x=0 then R23 denotes R27 and that when z=0 then R25 denotes R29.

The alkyl groups R22 may be linear or branched, preferably linear. Preferably, R22 denotes a methyl, ethyl, hydroxyethyl or dihydroxypropyl group, and more particularly a methyl or ethyl group.

Advantageously, the sum x+y+z is from 1 to 10.

When R23 is a hydrocarbon-based group R27, it may comprise from 12 to 22 carbon atoms, or else may comprise from 1 to 3 carbon atoms.

When R25 is a hydrocarbon-based group R29, it preferably contains 1 to 3 carbon atoms.

Advantageously, R24, R26 and R28, which may be identical or different, are chosen from linear or branched, saturated or unsaturated C₁₁-C₂₁ hydrocarbon-based groups, and more particularly from linear or branched C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X— is preferably a halide, preferably chloride, bromide or iodide, a (C₁-C₄)alkyl sulfate, a (C₁-C₄)alkylsulfonate or a (C₁-C₄)alkylarylsulfonate, a methanesulfonate, a phosphate, a nitrate, a tosylate, an anion derived from organic acid such as an acetate or a lactate or any other anion that is compatible with the ammonium bearing an ester function. The anion X— is more particularly a chloride, a methyl sulfate or an ethyl sulfate.

Use is more particularly made, in the composition according to the invention, of the ammonium salts of formula (IVa) in which:
R22 denotes a methyl or ethyl group,
x and y are equal to 1,
z is equal to 0 or 1,
r, s and t are equal to 2,
R23 is chosen from the group R26-C(═O)—; methyl, ethyl or C₁₄-C₂₂ hydrocarbon-based groups, and a hydrogen atom,
R25 is chosen from the group R28-C(═O)—; and a hydrogen atom,
R24, R26 and R28, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Among the compounds of formula (Va), mention may be made of the salts, notably the chloride or methyl sulfate of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures notably of plant or animal origin, or by transesterification of the methyl esters thereof. This esterification may be followed by quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin. Such compounds are sold, for example, under the names Dehyquart® by the company Henkel, Stepanquat® by the company Stepan, Noxamium® by the company CECA or Rewoquat® WE 18 by the company Evonik.

The composition according to the invention may contain, for example, a mixture of quaternary ammonium monoester, diester and triester salts with a weight majority of diester salts. Use may also be made of the ammonium salts containing at least one ester function that are described in U.S. Pat. Nos. 4,874,554 and 4,137,180. Use may also be made of the behenoylhydroxypropyltrimethylammonium chloride sold, for example, by the company Kao under the name Quartamin BTC 131.

Preferably, the ammonium salts containing at least one ester function contain two ester functions.

Mention may be made, as fatty amines, of amidoamines.

The amidoamines according to the invention can be chosen from fatty amidoamines, it being possible for the fatty chain to be borne by the amine group or by the amido group.

The term “amidoamine” is understood to mean a compound comprising at least one amide function and at least one primary, secondary or tertiary amine function.

The term “fatty amidoamine” is understood to mean an amidoamine comprising, generally, at least one C6-C30 hydrocarbon-based chain. Preferably, the fatty amidoamines of use according to the invention are not quaternized.

Preferably, the fatty amidoamines of use according to the invention are not (poly)oxyalkylenated.

Mention may be made, among the fatty amidoamines of use according to the invention, of the amidoamines of the following formula (Va):

RCONHR″N(R′)2 (Va)
in which:
   R represents a linear or branched, saturated or unsaturated and substituted or unsubstituted monovalent hydrocarbon-based radical having from 5 to 29 carbon atoms, preferably from 7 to 23 carbon atoms, and in particular a linear or branched C5-C29, preferably C7-C23, alkyl radical or a linear or branched C5-C29, preferably C7-C23, alkenyl radical;
   R″ represents a divalent hydrocarbon-based radical having less than 6 carbon atoms, preferably from 2 to 4 carbon atoms, better still 3 carbon atoms; and
   R′, which may be identical or different, represent a linear or branched, saturated or unsaturated and substituted or unsubstituted monovalent hydrocarbon-based radical having less than 6 carbon atoms, preferably from 1 to 4 carbon atoms, preferably a methyl radical.

The fatty amidoamines of formula (Va) are, for example, chosen from oleamidopropyldimethylamine, stearamidopropyldimethylamine, sold by Inolex Chemical Company under the name Lexamine S13, isostearamidopropyldimethylamine, stearamidoethyldimethylamine, lauramidopropyldimethylamine, myristamidopropyldimethylamine, behenamidopropyldimethylamine, dilinoleamidopropyldimethylamine, palmitamidopropyldimethylamine, ricinoleamidopropyldimethylamine, soyamidopropyldimethylamine, avocadoamidopropyldimethylamine, cocamidopropyldimethylamine, minkamidopropyldimethylamine, oatamidopropyldimethylamine, sesamidopropyldimethylamine, tallamidopropyldimethylamine, olivamidopropyldimethylamine, palmitamidopropyldimethylamine, stearamidoethyldiethylamine, brassicamidopropyldimethylamine and mixtures thereof.

Preferably, the fatty amidoamines are chosen from oleamidopropyldimethylamine, behenamidopropyldimethylamine, stearamidopropyldimethylamine, brassicamidopropyldimethylamine and mixtures thereof.

Preferably, the cationic surfactants i) are chosen from those of formula (Ia), (IVa) or (Va) and better still from cetyltrimethylammonium, behenyltrimethylammonium, dipalmitoylethylhydroxyethylmethylammonium salts and mixtures thereof; and more particularly from behenyltrimethylammonium chloride or methosulfate, cetyltrimethylammonium chloride or methosulfate, dipalmitoylethylhydroxyethylmethylammonium chloride or methosulfate, oleamidopropyldimethylamine, behenamidopropyldimethylamine, stearamidopropyldimethylamine, brassicamidopropyldimethylamine and mixtures thereof.

The total content of the cationic surfactant(s) i) present in the solid composition according to the invention preferably ranges from 0.01% to 15% by weight, preferentially from 0.1% to 10% by weight, better still from 0.5% to 8% by weight, and even better still from 1% to 5% by weight relative to the total weight of the composition.

### Starch(es) ii)

The solid composition according to the present invention comprises one or more starches ii).

The starch molecules which may be used in the present invention may originate from any plant source of starch, notably cereals and tubers; more particularly, they may be starches from corn, rice, cassava, barley, potato, wheat, sorghum, pea, oat or tapioca. It is also possible to use hydrolysates of the starches mentioned above. The starch is preferably derived from corn or potato.

The starches may be chemically or physically modified, notably by one or more of the following reactions: pregelatinization, oxidation, crosslinking, esterification, etherification, amidation, heat treatments.

More particularly, these reactions may be performed in the following manner:
pregelatinization by splitting the starch granules (for example drying and cooking in a drying drum);
oxidation with strong oxidizing agents, leading to the introduction of carboxyl groups into the starch molecule and to depolymerization of the starch molecule (for example by treating an aqueous starch solution with sodium hypochlorite);
crosslinking with functional agents capable of reacting with the hydroxyl groups of the starch molecules, which will thus be bonded together (for example with glyceryl and/or phosphate groups);
esterification in alkaline medium for the grafting of functional groups, notably C₁ to C₆ acyl (acetyl), C₁ to C₆ hydroxyalkyl (hydroxyethyl or hydroxypropyl), carboxymethyl or octenylsuccinic.

Monostarch phosphates (of the type St-O—PO—(OX)₂), distarch phosphates (of the type St-O—PO—(OX)—O-St) or even tristarch phosphates (of the type St-O—PO—(O-St)₂) or mixtures thereof may notably be obtained by crosslinking with phosphorus compounds; with St meaning starch and X notably denoting alkali metals (for example sodium or potassium), alkaline-earth metals (for example calcium or magnesium), ammonia salts, amine salts such as salts of monoethanolamine, diethanolamine, triethanolamine or 3-amino-1,2-propanediol, and ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

Distarch phosphates may notably be mentioned, for instance the product sold under the references Prejel VA-70-T AGGL (gelatinized hydroxypropyl cassava distarch phosphate), Prejel TK1 (gelatinized cassava distarch phosphate) and Prejel 200 (gelatinized acetylated cassava distarch phosphate) by the company Avebe, or Structure Zea from National Starch (gelatinized corn distarch phosphate).

A preferred starch is a starch that has undergone at least one chemical modification such as at least one esterification.

According to the invention, use may also be made of amphoteric starches, comprising one or more anionic groups and one or more cationic groups. The anionic and cationic groups may be bonded to the same reactive site of the starch molecule or to different reactive sites; they are preferably bonded to the same reactive site. The anionic groups may be of carboxylic, phosphate or sulfate type, preferably carboxylic. The cationic groups may be of primary, secondary, tertiary or quaternary amine type.

The amphoteric starches are notably chosen from the compounds having the following formulae:

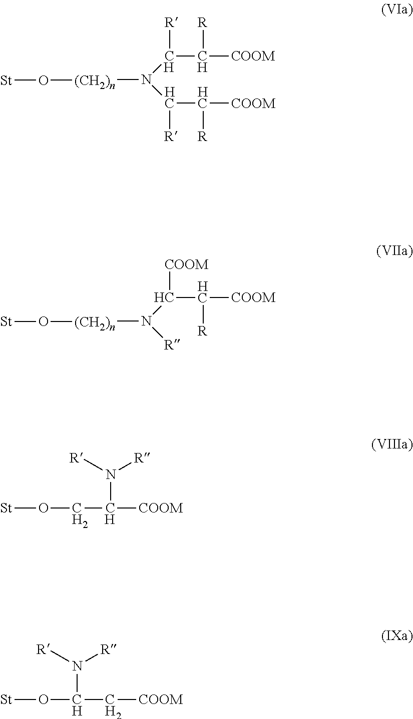

in which formulae (VIa) to (IXa):
   St-O represents a starch molecule;
   R, which may be identical or different, represents a hydrogen atom or a methyl radical;
   R′, which may be identical or different, represents a hydrogen atom, a methyl radical or a —C(O)—OH group;
   n is an integer equal to 2 or 3;
   M, which may be identical or different, denotes a hydrogen atom, an alkali metal or alkaline-earth metal such as Na, K or Li, a quaternary ammonium NH₄, or an organic amine; and
   R″ represents a hydrogen atom or a C₁-C₁₈ alkyl radical.

These compounds are notably described in U.S. Pat. Nos. 5,455,340 and 4,017,460.

Starches of formula (VIIa) or (VIIIa), and preferentially starches modified with 2-chloroethylaminodipropionic acid are particularly used, i.e. starches of formula (VIIa) or (VIIIa) in which R, R′, R″ and M represent a hydrogen atom and n is equal to 2. Preferably, the amphoteric starch is a starch chloroethylamido dipropionate.

More particularly, the starches are chosen from maize starches, potato starches, rice starches and modified starches such as those mentioned above, notably phosphate starches such as distarch phosphates such as those described above.

In one embodiment, the solid composition comprises at least two starches. More particularly, the solid composition comprises at least one unmodified starch (or native starch) such as a maize starch or a potato starch or a rice starch, and at least one modified starch in particular chosen from those described above, and more particularly from the phosphate starches mentioned above, such as distarch phosphates.

More preferentially still, said starches comprise at least one maize, rice or potato starch, and at least one modified starch such as a phosphate starch.

The total content of the starch(es) ii) present in the solid composition according to the invention preferably ranges from 10% to 90% by weight, preferentially from 20% to 85% by weight, better still from 30% to 80% by weight, and even better still from 40% to 75% by weight, relative to the total weight of the composition.

In particular, the total content of unmodified starch(es) present in the solid composition according to the invention preferably ranges from 10% to 85% by weight, preferentially from 20% to 80% by weight, better still from 30% to 75% by weight, relative to the total weight of the composition.

In particular, the total content of modified starch(es), especially phosphate starch(es), present in the solid composition according to the invention preferably ranges from 0.1% to 30% by weight, preferentially from 0.5% to 20% by weight, and better still from 2% to 15% by weight, relative to the total weight of the composition.

Amphoteric or Zwitterionic Surfactant(s) iii)

The solid composition according to the invention comprises one or more amphoteric or zwitterionic surfactants iii).

In particular, the amphoteric or zwitterionic surfactant(s), which are preferably non-silicone, used in the solid composition according to the present invention may notably be derivatives of optionally quaternized secondary or tertiary aliphatic amines, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₁-C₆)alkylbetaines and (C₈-C₂₀)alkylamido(C₁-C₆)alkylsulfobetaines, and mixtures thereof.

Among the optionally quaternized derivatives of secondary or tertiary aliphatic amines that may be used, as defined above, mention may also be made of the compounds having the respective structures (Xa) and (XIa) below:

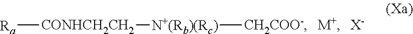

in which formula (Xa):
   Rₐ represents a C₁₀ to C₃₀ alkyl or alkenyl group derived from an acid RₐCOOH preferably present in hydrolysed coconut kernel oil; preferably, Rₐ represents a heptyl, nonyl or undecyl group;
   R_{b} represents a β-hydroxyethyl group;
   R_{c} represents a carboxymethyl group;
   M⁺ represents a cationic counterion derived from an alkali metal or alkaline-earth metal, such as sodium, an ammonium ion or an ion derived from an organic amine; and
   X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

Rₐ′—CONHCH₂CH₂—N(B)(B′) (XIa)
in which formula (XIa):
   B represents the group —CH₂CH₂OX′;
   B′ represents the group —(CH₂)_{z}Y′, with z=1 or 2;
   X′ represents the group —CH₂COOH, —CH₂—COOZ′, —CH₂CH₂COOH or CH₂CH₂—COOZ′, or a hydrogen atom;
   Y′ represents the group —COOH, —COOZ′ or —CH₂CH(OH)SO₃H or the group CH₂CH(OH)SO₃—Z′;
   Z′ represents a cationic counterion derived from an alkali metal or alkaline-earth metal, such as sodium, an ammonium ion or an ion derived from an organic amine;
   R_{a′} represents a C₁₀ to C₃₀ alkyl or alkenyl group of an acid R_{a′}—COOH which is preferably present in coconut kernel oil or in hydrolysed linseed oil, preferably R_{a′} an alkyl group, notably a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (XIIa):

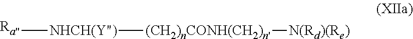

in which formula (XIIa):
   Y″ represents the group —COOH, —COOZ″ or —CH₂—CH(OH)SO₃H or the group CH₂CH(OH)SO₃—Z″;
   R_{d} and Rₑ, independently of each other, represent a C₁ to C₄ alkyl or hydroxyalkyl radical;
   Z″ represents a cationic counterion derived from an alkali metal or alkaline-earth metal, such as sodium, an ammonium ion or an ion derived from an organic amine;
   R_{a″} represents a C₁₀ to C₃₀ alkyl or alkenyl group of an acid R_{a″}—COOH which is preferably present in coconut kernel oil or in hydrolysed linseed oil; and
   n and n′ denote, independently of each other, an integer ranging from 1 to 3.

Among the compounds of formula (XIIa), mention may be made of the compound classified in the CTFA dictionary under the name sodium diethylaminopropyl cocoaspartamide and sold by the company Chimex under the name Chimexane HB.

These compounds may be used alone or as mixtures.

Among the amphoteric or zwitterionic surfactants mentioned above, use is advantageously made of (C₈-C₂₀)alkylbetaines, such as cocoyl betaine, (C₈-C₂₀)alkylamido(C₃-C₈)alkylbetaines, such as cocamidopropylbetaine, (C₈-C₂₀)alkylamphoacetates, (C₈-C₂₀)alkylamphodiacetates and mixtures thereof; and preferably (C₈-C₂₀)alkylbetaines, (C₈-C₂₀)alkylamido(C₃-C₈)alkylbetaines and mixtures thereof.

Preferentially, the amphoteric or zwitterionic surfactant(s) are chosen from (C₈-C₂₀)alkylbetaines, (C₈-C₂₀)alkylamido(C₃-C₈)alkylbetaines and mixtures thereof, and better still from (C₈-C₂₀)alkylamido(C₃-C₈)alkylbetaines and mixtures thereof.

The solid composition according to the invention comprises a total content of the amphoteric or zwitterionic surfactant(s) iii) which ranges from 0.01% to 10% by weight, more preferentially from 0.05% to 5% by weight, and even better still from 0.1% to 1% by weight, relative to the total weight of the composition.

Advantageously, the solid composition according to the invention comprises a total content of amphoteric or zwitterionic surfactants chosen from (C₈-C₂₀)alkylbetaines, (C₈-C₂₀)alkylamido(C₃-C₈)alkylbetaines and mixtures thereof which preferably ranges from 0.01% to 10% by weight, more preferentially from 0.05% to 5% by weight, and even better still from 0.1% to 1% by weight, relative to the total weight of the composition.

### Fatty Substances iv)

The solid composition according to the invention comprises one or more, preferably liquid, fatty substances iv).

The term “fatty substance” means an organic compound that is insoluble in water at 25 °C and at atmospheric pressure (1.013×105 Pa) (solubility of less than 5% by weight, preferably less than 1% by weight and even more preferentially less than 0.1% by weight). The fatty substances have in their structure at least one hydrocarbon-based chain including at least six carbon atoms and/or a sequence of at least two siloxane groups. In addition, the fatty substances are generally soluble in organic solvents under the same temperature and pressure conditions, such as chloroform, dichloromethane, carbon tetrachloride, ethanol, benzene, toluene, tetrahydrofuran (THF), liquid petroleum jelly or decamethylcyclopentasiloxane.

The fatty substances usable in the present invention are neither (poly)oxyalkylenated nor (poly)glycerolated.

The term “non-silicone fatty substance” means a fatty substance not containing any Si—O bonds and the term “silicone fatty substance” means a fatty substance containing at least one Si—O bond.

The fatty substances useful according to the invention can be liquid fatty substances (or oils) and/or solid fatty substances. The term “liquid fatty substance” means a fatty substance having a melting point of less than or equal to 25 °C at atmospheric pressure (1.013×10⁵ Pa). The term “solid fatty substance” means a fatty substance having a melting point of greater than 25 °C at atmospheric pressure (1.013×10⁵ Pa).

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name MDSC 2920 by the company TA Instruments. In the present patent application, all of the melting points are determined at atmospheric pressure (1.013×10⁵ Pa).

More particularly, the liquid fatty substance(s) according to the invention may be chosen from C6 to C16 liquid hydrocarbons, liquid hydrocarbons comprising more than 16 carbon atoms, non-silicone oils of animal origin, oils of triglyceride type of vegetable or synthetic origin, fluoro oils, liquid fatty alcohols, liquid esters of fatty acid and/or of fatty alcohol other than triglycerides, silicone oils, and mixtures thereof.

It is recalled that the fatty alcohols, esters and acids more particularly contain at least one saturated or unsaturated, linear or branched hydrocarbon-based group, comprising from 6 to 40 and better still from 8 to 30 carbon atoms, which is optionally substituted, in particular, with one or more hydroxyl groups (in particular 1 to 4). If they are unsaturated, these compounds may comprise one to three conjugated or unconjugated carbon-carbon double bonds.

As regards the C6 to C16 liquid hydrocarbons, these may be linear, branched, or optionally cyclic, and are preferably chosen from alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, and of mineral or synthetic origin, and are preferably chosen from liquid paraffins or liquid petroleum jelly, polydecenes, hydrogenated polyisobutene such as Parleam®, and mixtures thereof.

A hydrocarbon-based oil (or non-silicone oil) of animal origin that may be mentioned is perhydrosqualene.

The triglyceride oils of vegetable or synthetic origin are preferably chosen from liquid triglycerides of fatty acids including from 6 to 30 carbon atoms, for instance heptanoic or octanoic acid triglycerides, or alternatively, for example, sunflower, maize, soybean, marrow, grapeseed, sesame, hazelnut, apricot, macadamia, arara, castor and avocado oils, caprylic/capric acid triglycerides, for instance those sold by the company Stéarinerie Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethylperfluoromorpholine sold under the name PF 5052® by the company 3M.

The liquid fatty alcohols that are suitable for use in the invention are more particularly chosen from linear or branched, saturated or unsaturated alcohols, preferably unsaturated or branched alcohols, including from 6 to 40 carbon atoms and preferably from 8 to 30 carbon atoms. These fatty alcohols are neither oxyalkylenated nor glycerolated. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof. Preferably, oleyl alcohol will be used.

As regards the liquid esters of fatty acids and/or of fatty alcohols other than the triglycerides mentioned above, mention may be made notably of esters of saturated or unsaturated, linear C1 to C26 or branched C3 to C26 aliphatic monoacids or polyacids and of saturated or unsaturated, linear C1 to C26 or branched C3 to C26 aliphatic monoalcohols or polyalcohols, the total carbon number of the esters being greater than or equal to 6 and more advantageously greater than or equal to 10.

Preferably, for the esters of monoalcohols, at least one from among the alcohol and the acid is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl and isopropyl palmitates, such as 2-ethylhexyl palmitate, 2-octyldecyl palmitate, alkyl myristates such as isopropyl myristate; isobutyl stearate; 2-hexyldecyl laurate, and mixtures thereof.

Preferably, among the monoesters of monoacids and of monoalcohols, use will be made of ethyl palmitate and isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Esters of C4 to C22 dicarboxylic or tricarboxylic acids and of C1 to C22 alcohols and esters of mono-, di- or tricarboxylic acids and of C2 to C26 di-, tri-, tetra- or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C6 to C30, preferably C12 to C22, fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides other than the anionic polysaccharides as described below.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described previously and of linear or branched, saturated or unsaturated C6 to C30 and preferably C12 to C22 fatty acids.

If they are unsaturated, these compounds may comprise one to three conjugated or unconjugated carbon-carbon double bonds.

The esters may also be chosen from monoesters, diesters, triesters, tetraesters and polyesters, and mixtures thereof.

These esters may be, for example, oleates, laurates, palmitates, myristates, behenates, cocoates, stearates, linoleates, linolenates, caprates, arachidonates or mixtures thereof notably such as the mixed oleo-palmitate, oleo-stearate and palmito-stearate esters.

More particularly, use is made of monoesters and diesters and notably sucrose, glucose or methylglucose mono- or di-oleates, -stearates, -behenates, -oleopalmitates, -linoleates, -linolenates and -oleostearates, and mixtures thereof.

An example that may be mentioned is the product sold under the name Glucate® DO by the company Amerchol, which is a methylglucose dioleate.

The silicone oils that may be used in the composition according to the present invention may be volatile or nonvolatile, cyclic, linear or branched silicone oils, which are unmodified or modified with organic groups, and preferably have a viscosity from 5×10-6 to 2.5 m²/s at 25 °C, and preferably 1×10-5 to 1 m²/s.

Preferably, the silicone oils are chosen from polydialkylsiloxanes, notably polydimethylsiloxanes (PDMS), and liquid polyorganosiloxanes including at least one aryl group.

These silicone oils may also be organomodified. The organomodified silicone oils that may be used in accordance with the invention are preferably liquid silicones as defined previously and including in their structure one or more organofunctional groups attached via a hydrocarbon-based group, chosen, for example, from amine groups and alkoxy groups.

Organopolysiloxanes are defined in greater detail in Walter Noll's Chemistry and Technology of Silicones (1968), Academic Press. They may be volatile or nonvolatile.

When they are volatile, the silicone oils are more particularly chosen from those with a boiling point of between 60 °C and 260 °C, and even more particularly from:
(i) cyclic polydialkylsiloxanes including from 3 to 7 and preferably from 4 to 5 silicon atoms. These are, for example, octamethylcyclotetrasiloxane sold notably under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.

Mention may also be made of cyclocopolymers of the dimethylsiloxane/methylalkylsiloxane type, such as Volatile Silicone® FZ 3109 sold by the company Union Carbide.

Mention may also be made of mixtures of cyclic polydialkylsiloxanes with silicon-derived organic compounds, such as the mixture of octamethylcyclotetrasiloxane and tetra(trimethylsilyl)pentaerythritol (50/50) and the mixture of octamethylcyclotetrasiloxane and 1,1′-oxy(2,2,2′,2′,3,3′-hexatrimethylsilyloxy)bisneopentane;
(ii) linear volatile polydialkylsiloxanes containing 2 to 9 silicon atoms and having a viscosity of less than or equal to 5×10−6 m²/s at 25 °C. An example is decamethyltetrasiloxane notably sold under the name SH 200 by the company Toray Silicone. Silicones falling within this category are also described in the article published in Cosmetics and Toiletries, Vol. 91, January 76, pages 27-32, Todd & Byers Volatile Silicone Fluids for Cosmetics.

Nonvolatile polydialkylsiloxanes are preferably used.

These silicone oils are more particularly chosen from polydialkylsiloxanes, among which mention may be made mainly of polydimethylsiloxanes bearing trimethylsilyl end groups. The viscosity of the silicones is measured at 25 °C in accordance with the standard ASTM 445 Appendix C.

Among these polydialkylsiloxanes, mention may be made non-limitingly of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

The organomodified silicones that may be used in accordance with the invention are silicones as defined previously and including in their structure one or more organofunctional groups attached via a hydrocarbon-based group.

As regards the liquid polyorganosiloxanes including at least one aryl group, these may notably be polydiphenylsiloxanes, and polyalkylarylsiloxanes functionalized with the organofunctional groups mentioned above.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25 °C.

Among these polyalkylarylsiloxanes, mention may be made by way of example of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Among the organomodified silicones, mention may be made of organopolysiloxanes including:
substituted or unsubstituted amine groups, such as the products sold under the names GP 4 Silicone Fluid and GP 7100 by the company Genesee, or the products sold under the names Q2 8220 and Dow Corning 929 or 939 by the company Dow Corning. The substituted amine groups are in particular C₁ to C₄ aminoalkyl groups;
alkoxylated groups,
hydroxyl groups.

The silicones that may be used are preferably amino silicones. The term “amino silicone” denotes any silicone including at least one primary, secondary or tertiary amine or a quaternary ammonium group.

The weight-average molecular masses of these amino silicones may be measured by gel permeation chromatography (GPC) at room temperature (25 °C), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

Preferably, the amino silicone(s) that may be used in the context of the invention are chosen from:
a) the polysiloxanes corresponding to formula (A):

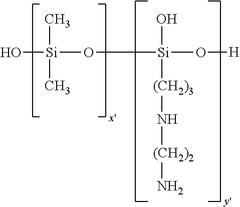

in which x″ and y′ are integers such that the weight-average molecular weight (Mw) is between 5000 and 500 000 approximately;
b) the amino silicones corresponding to formula (B):

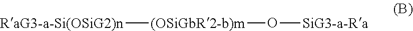

in which:
   G, which may be identical or different, denotes a hydrogen atom or a group from among phenyl, OH, C₁-C₈ alkyl, for example methyl, or C₁-C₈ alkoxy, for example methoxy,
   a, which may be identical or different, denotes 0 or an integer from 1 to 3, in particular 0,
   b denotes 0 or 1, in particular 1,
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10,
   R′, which may be identical or different, denotes a monovalent radical of formula —CqH2qL in which q is a number ranging from 2 to 8 and L is an optionally quaternized amine group chosen from the following groups:
   —N(R″)2; —N+(R″)3 A-; —NR″-Q-N(R″)2 and —NR″-Q-N+(R″)3 A-,
in which R″, which may be identical or different, denotes hydrogen, phenyl, benzyl, or a saturated monovalent hydrocarbon-based radical, for example a C₁-C₂₀ alkyl radical; Q denotes a linear or branched group of formula CrH2r, r being an integer ranging from 2 to 6, preferably from 2 to 4; and A⁻ represents a cosmetically acceptable anion, notably a halide such as fluoride, chloride, bromide or iodide.

Preferably, the amino silicones are chosen from the amino silicones of formula (B). Preferably, the amino silicones of formula (B) are chosen from the amino silicones corresponding to formulae (C), (D), (E), (F), (G) and/or (K) below.

Thus, the amino silicones corresponding to formula (B) may be chosen from, alone or as a mixture:

A/ the silicones known as “trimethylsilyl amodimethicone” corresponding to formula (C):

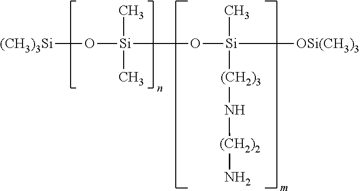

in which m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10.

B/ the silicones of formula (D) below:

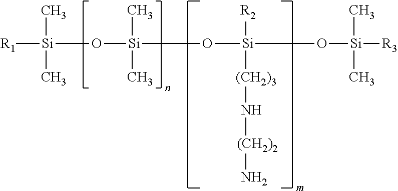

in which:
   m and n are numbers such that the sum (n+m) ranges from 1 to 1000, in particular from 50 to 250 and more particularly from 100 to 200; n possibly denoting a number from 0 to 999, notably from 49 to 249 and more particularly from 125 to 175, and m possibly denoting a number from 1 to 1000, notably from 1 to 10 and more particularly from 1 to 5;
   R₁, R₂ and R₃, which may be identical or different, represent a hydroxyl or C₁-C₄ alkoxy radical, at least one of the radicals R₁ to R₃ denoting an alkoxy radical.

Preferably, the alkoxy radical is a methoxy radical.

The hydroxy/alkoxy mole ratio preferably ranges from 0.2:1 to 0.4:1 and preferably from 0.25:1 to 0.35:1 and more particularly is equal to 0.3:1.

The weight-average molecular mass (Mw) of these silicones preferably ranges from 2000 to 1 000 000 and more particularly from 3500 to 200 000.

C/ the silicones of formula (E) below:

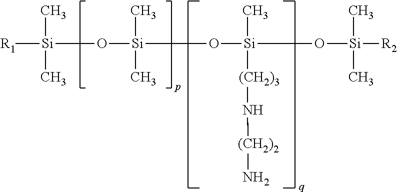

in which:
   p and q are numbers such that the sum (p+q) ranges from 1 to 1000, in particular from 50 to 350 and more particularly from 150 to 250; p possibly denoting a number from 0 to 999, notably from 49 to 349 and more particularly from 159 to 239, and q possibly denoting a number from 1 to 1000, notably from 1 to 10 and more particularly from 1 to 5;
   R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly is equal to 1:0.95.

The weight-average molecular mass (Mw) of the silicone preferably ranges from 2000 to 200 000, even more particularly from 5000 to 100 000 and more particularly from 10 000 to 50 000.

The commercial products comprising silicones of structure (D) or (E) may include in their composition one or more other amino silicones the structure of which is different from formula (D) or (E).

A product containing amino silicones of structure (D) is sold by the company Wacker under the name Belsil® ADM 652.

A product containing amino silicones of structure (E) is sold by Wacker under the name Fluid WR 1300®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nanometers. Preferably, notably as amino silicones of formula (E), use is made of microemulsions with a mean particle size ranging from 5 nm to 60 nanometres (limits included) and more particularly from 10 nm to 50 nanometres (limits included). Thus, use may be made according to the invention of the amino silicone microemulsions of formula (E) sold under the names Finish CT 96 E® or SLM 28020® by the company Wacker.

D/ the silicones of formula (F) below:

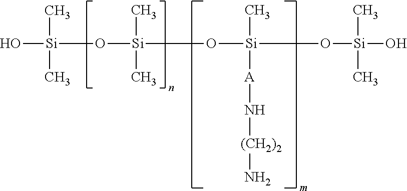

in which:
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
   A denotes a linear or branched alkylene radical containing from 4 to 8 carbon atoms and preferably 4 carbon atoms. This radical is preferably linear.

The weight-average molecular mass (Mw) of these amino silicones preferably ranges from 2000 to 1 000 000 and even more particularly from 3500 to 200 000.

A silicone corresponding to this formula is, for example, the Xiameter MEM 8299 Emulsion from Dow Corning.

E/ the silicones of formula (G) below:

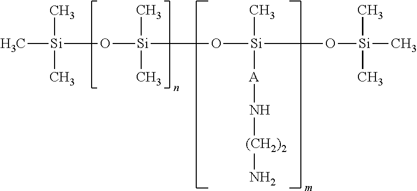

in which:
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
   A denotes a linear or branched alkylene radical containing from 4 to 8 carbon atoms and preferably 4 carbon atoms. This radical is preferably branched.

The weight-average molecular mass (Mw) of these amino silicones preferably ranges from 500 to 1 000 000 and even more particularly from 1000 to 200 000.

A silicone corresponding to this formula is, for example, DC2-8566 Amino Fluid from Dow Corning;
c) the amino silicones corresponding to formula (H):

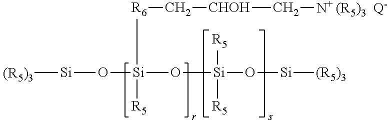

in which:
   R₅ represents a monovalent hydrocarbon-based radical containing from 1 to 18 carbon atoms, and in particular a C₁-C₁₈ alkyl or C₂-C₁₈ alkenyl radical, for example methyl;
   R₆ represents a divalent hydrocarbon-based radical, notably a C₁-C₁₈ alkylene radical or a divalent C₁-C₁₈, for example C₁-C₈, alkyleneoxy radical linked to the Si via an SiC bond;
   Q⁻ is an anion such as a halide ion, notably chloride, or an organic acid salt, notably acetate;
   r represents a mean statistical value ranging from 2 to 20 and in particular from 2 to 8;
   s represents a mean statistical value ranging from 20 to 200 and in particular from 20 to 50.

Such amino silicones are notably described in patent U.S. Pat. No. 4,185,087.
d) the quaternary ammonium silicones of formula (I):

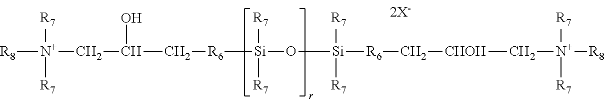

in which:
   R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;
   R₆ represents a divalent hydrocarbon-based radical, notably a C₁-C₁₈ alkylene radical or a divalent C₁-C₁₈, for example C₁-C₈, alkyleneoxy radical linked to the Si via an SiC bond;
   R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R6-NHCOR7;
   X⁻ is an anion such as a halide ion, notably chloride, or an organic acid salt, notably acetate;
   r represents a mean statistical value ranging from 2 to 200 and in particular from 5 to 100.

These silicones are described, for example, in patent application EP-A 0 530 974;
e) the amino silicones of formula (J):

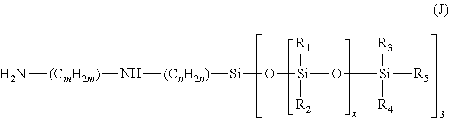

in which:
   R₁, R₂, R₃ and R₄, which may be identical or different, denote a C₁-C₄ alkyl radical or a phenyl group,
   R₅ denotes a C₁-C₄ alkyl radical or a hydroxyl group,
   n is an integer ranging from 1 to 5,
   m is an integer ranging from 1 to 5, and
   x is chosen such that the amine number ranges from 0.01 to 1 meq/g;
f) multiblock polyoxyalkylene amino silicones, of the type (AB)n, A being a polysiloxane block and B being a polyoxyalkylene block including at least one amine group.

Said silicones are preferably formed from repeating units having the following general formulae:

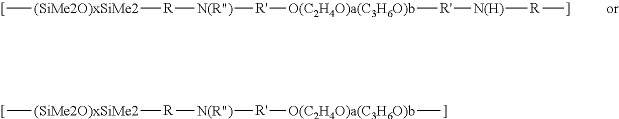

in which:
   a is an integer greater than or equal to 1, preferably ranging from 5 to 200 and more particularly ranging from 10 to 100;
   b is an integer between 0 and 200, preferably ranging from 4 to 100 and more particularly between 5 and 30;
   x is an integer ranging from 1 to 10 000 and more particularly from 10 to 5000;
   R″ is a hydrogen atom or a methyl;
   R, which may be identical or different, represent a linear or branched divalent C₂-C₁₂ hydrocarbon-based radical, optionally including one or more heteroatoms such as oxygen; preferably, R denotes an ethylene radical, a linear or branched propylene radical, a linear or branched butylene radical or a CH₂CH₂CH₂OCH₂CH(OH)CH₂— radical; preferentially, R denotes a CH₂CH₂CH₂OCH₂CH(OH)CH₂— radical;
   R′, which may be identical or different, represent a linear or branched divalent C₂-C₁₂ hydrocarbon-based radical, optionally including one or more heteroatoms such as oxygen; preferably, R′ denotes an ethylene radical, a linear or branched propylene radical, a linear or branched butylene radical or a radical CH₂CH₂CH₂OCH₂CH(OH)CH₂—; preferentially, R′ denotes —CH(CH₃)—CH₂—.

The siloxane blocks preferably represent between 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1 000 000 and more particularly between 10 000 and 200 000.

Mention may notably be made of the silicones sold under the name Silsoft A-843 or Silsoft A+ by Momentive.
g) α,ω-bis-amino silicones corresponding to the following formula (K)

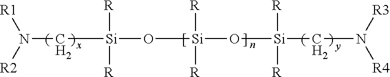

in which:
   the radicals R, independently of each other, represent a hydrogen atom, an OH group or a linear or branched C1-C4 alkyl group;
   the radicals R1, R2, R3 and R4, independently of each other, represent a hydrogen atom, a C1-C6 alkyl group or a C1-C6 aminoalkyl group;
   x is between 0 and 6; y is between 0 and 6, and
   n is such that the weight-average molecular mass (Mw) of the amino silicone is between 5000 and 200 000 g/mol.

Preferably, the radicals R are identical and represent CH3 (methyl).

Preferably, R1, R2, R3 and R4, independently of each other, represent a hydrogen atom, an alkyl group, preferably a saturated linear C1-C4 and better still C2-C4 alkyl group, notably ethyl; or a C2-C4 aminoalkyl group, notably having the structure —(CaH2a)-NH2 with a=2 to 4; in particular aminoethyl (—CH2-CH2-NH2).

Preferably, x is between 1 and 5, better still between 2 and 4, even better still x=3.

Preferably, y is between 1 and 5, better still between 2 and 4, even better still y=3.

Preferably, x=y.

Preferably, n is such that the weight-average molecular mass (Mw) of the silicone is between 10 000 and 150 000 g/mol, or even between 15 000 and 100 000 g/mol.

More preferentially, the amino silicone corresponds to the formula (K) in which the radicals R represent a methyl group, x=y=3 and R1, R2, R3 and R4 represent a hydrogen atom; it is then a bis-aminopropyl dimethicone (INCI name).

Preferably, the liquid fatty substance(s) are chosen from vegetable oils such as those defined above, liquid fatty esters such as those defined above, amino silicones such as amodimethicone and bis-amino silicones, and mixtures thereof.

The solid fatty substances preferably have a viscosity of greater than 2 Pa·s, measured at 25 °C and at a shear rate of 1 s⁻¹.

The solid fatty substance(s) are preferably chosen from solid fatty alcohols, solid esters of fatty acids and/or of fatty alcohols, waxes, ceramides, and mixtures thereof.

The term “fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated.

The solid fatty alcohols may be saturated or unsaturated, and linear or branched, and include from 8 to 40 carbon atoms, preferably from 10 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon atoms, or even from 12 to 24 atoms, and even better still from 14 to 22 carbon atoms.

The solid fatty alcohols that may be used are preferably chosen from saturated or unsaturated, linear or branched, preferably linear and saturated, (mono)alcohols including from 8 to 40 carbon atoms, better still from 10 to 30, or even from 12 to 24 atoms, and even better still from 14 to 22 carbon atoms.

The solid fatty alcohols that may be used may be chosen, alone or as a mixture, from: myristyl alcohol (or 1-tetradecanol); cetyl alcohol (or 1-hexadecanol); stearyl alcohol (or 1-octadecanol); arachidyl alcohol (or 1-eicosanol); behenyl alcohol (or 1-docosanol); lignoceryl alcohol (or 1-tetracosanol); ceryl alcohol (or 1-hexacosanol); montanyl alcohol (or 1-octacosanol); myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl or cetearyl alcohol. Particularly preferably, the solid fatty alcohol is cetylstearyl or cetearyl alcohol.

The solid esters of a fatty acid and/or of a fatty alcohol that may be used are preferably chosen from esters resulting from a C₉-C₂₆ carboxylic fatty acid and/or from a C₉-C₂₆ fatty alcohol.

Preferably, these solid fatty esters are esters of a linear or branched, saturated carboxylic acid including at least 10 carbon atoms, preferably from 10 to 30 carbon atoms and more particularly from 12 to 24 carbon atoms, and of a linear or branched, saturated monoalcohol including at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxylated alcohols may also be used.

Mention may notably be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C₉-C₂₆ alkyl palmitates, notably myristyl palmitate, cetyl palmitate or stearyl palmitate; C₉-C₂₆ alkyl myristates, such as cetyl myristate, stearyl myristate and myristyl myristate; C₉-C₂₆ alkyl stearates, notably myristyl stearate, cetyl stearate and stearyl stearate; and mixtures thereof.

For the purposes of the present invention, a wax is a lipophilic compound, which is solid at 25 °C and atmospheric pressure, with a reversible solid/liquid change of state, having a melting point greater than about 40 °C, which may be up to 200 °C, and having in the solid state anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may notably be made of hydrocarbon-based waxes, for instance beeswax, notably of organic origin, lanolin wax, and Chinese insect waxes; rice bran wax, carnauba wax, candelilla wax, ouricury wax, esparto grass wax, berry wax, shellac wax, Japan wax and sumac wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of C₂₀ to C₆₀ microcrystalline waxes, such as Microwax HW.

Mention may also be made of the MW 500 polyethylene wax sold under the reference Permalen 50-L polyethylene.

Mention may also be made of waxes obtained by catalytic hydrogenation of animal or plant oils containing linear or branched C₈ to C₃₂ fatty chains. Among these, mention may notably be made of isomerized jojoba oil, such as the trans-isomerized partially hydrogenated jojoba oil, notably the product manufactured or sold by the company Desert Whale under the commercial reference Iso-Jojoba-50®, hydrogenated sunflower oil, hydrogenated castor oil, hydrogenated coconut kernel oil, hydrogenated lanolin oil, and bis(1,1,1-trimethylolpropane) tetrastearate, notably the product sold under the name Hest 2T-4S® by the company Heterene.

The waxes obtained by hydrogenation of castor oil esterified with cetyl alcohol, such as the products sold under the names Phytowax Castor 16L64® and 22L73® by the company Sophim, may also be used.

A wax that may also be used is a C₂₀ to C₄₀ alkyl (hydroxystearyloxy)stearate (the alkyl group containing from 20 to 40 carbon atoms), alone or as a mixture. Such a wax is notably sold under the names “Kester Wax K 82 P®”, “Hydroxypolyester K 82 P®” and “Kester Wax K 80 P®” by the company Koster Keunen.

It is also possible to use microwaxes in the compositions of the invention; mention may notably be made of carnauba microwaxes, such as the product sold under the name MicroCare 350® by the company Micro Powders, synthetic-wax microwaxes, such as the product sold under the name MicroEase 114S® by the company Micro Powders, microwaxes constituted of a mixture of carnauba wax and of polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The waxes are preferably chosen from mineral waxes, for instance paraffin, petroleum jelly, lignite or ozokerite wax; plant waxes, for instance cocoa butter or cork fibre or sugar cane waxes, olive tree wax, rice wax, hydrogenated jojoba wax, ouricury wax, carnauba wax, candelilla wax, esparto grass wax, or absolute waxes of flowers, such as the essential wax of blackcurrant blossom sold by the company Bertin (France); waxes of animal origin, for instance beeswaxes or modified beeswaxes (Cera Bellina), spermaceti, lanolin wax and lanolin derivatives; microcrystalline waxes; and mixtures thereof.

The ceramides, or ceramide analogues such as glycoceramides, which may be used in the compositions according to the invention, are known; mention may be made in particular of ceramides of classes I, II, III and V according to the Dawning classification.

The ceramides or analogues thereof that may be used preferably correspond to the following formula: R³CH(OH)CH(CH₂OR²)(NHCOR¹), in which:
R¹ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R² denotes a hydrogen atom, a (glycosyl)n group, a (galactosyl)m group or a sulfogalactosyl group, in which n is an integer ranging from 1 to 4 and m is an integer ranging from 1 to 8;
R³ denotes a C₁₅-C₂₆ hydrocarbon-based group, which is saturated or unsaturated in the alpha position, it being possible for this group to be substituted with one or more C₁-C₁₄ alkyl groups; it being understood that in the case of natural ceramides or glycoceramides, R³ may also denote a C₁₅-C₂₆ alpha-hydroxyalkyl group, the hydroxyl group being optionally esterified with a C₁₆-C₃₀ alpha-hydroxy acid.

The ceramides that are more particularly preferred are the compounds for which R¹ denotes a saturated or unsaturated alkyl derived from C₁₆-C₂₂ fatty acids; R² denotes a hydrogen atom and R³ denotes a saturated linear C₁₅ group.

Preferentially, use is made of ceramides for which R¹ denotes a saturated or unsaturated alkyl group derived from C₁₄-C₃₀ fatty acids; R² denotes a galactosyl or sulfogalactosyl group; and R³ denotes a —CH═CH—(CH₂)₁₂—CH₃ group.

Use may also be made of the compounds for which R¹ denotes a saturated or unsaturated alkyl radical derived from C₁₂-C₂₂ fatty acids; R² denotes a galactosyl or sulfogalactosyl radical; and R³ denotes a saturated or unsaturated C₁₂-C₂₂ hydrocarbon-based radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. N-Oleoyldihydrosphingosine will preferably be used.

The solid fatty substances are preferably chosen from solid fatty alcohols, in particular from cetyl alcohol, stearyl alcohol and mixtures thereof such as cetylstearyl or cetearyl alcohol.

Butters may also be used.

For the purposes of the present invention, the term “butter” (also referred to as a “pasty fatty substance”) means a lipophilic fatty compound with a reversible solid/liquid change of state, comprising at a temperature of 25 °C and at atmospheric pressure (760 mmHg) a liquid fraction and a solid fraction. Preferably, the butter(s) according to the invention have a melting start temperature of greater than 25 °C and a melting end temperature of less than 60 °C.

Preferably, the particular butter(s) are of plant origin, such as those described in Ullmann's Encyclopedia of Industrial Chemistry (“Fats and Fatty Oils”, A. Thomas, published online: 15 Jun. 2000, DOI: 10.1002/14356007.a10_173, point 13.2.2.2. Shea Butter, Borneo Tallow, and Related Fats (Vegetable Butters)).

Mention may be made more particularly of shea butter, Nilotica shea butter (Butyrospermum parkii), galam butter, (Butyrospermum parkii), Borneo butter or fat or tengkawang tallow (Shorea stenoptera), Shorea butter, illipé butter, Madhuca butter or bassia butter (Madhuca longifolia), mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya butter (Kpangnan) (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter and sunflower butter.

An example of a preferred butter is shea butter.

In a known manner, shea butter is extracted from the fruits (also called “kernels” or “nuts”) of the Butyrospermum parkii tree. Each fruit contains between 45% and 55% fatty substance, which is generally extracted and refined.

According to a preferred embodiment, the composition according to the invention comprises one or more liquid fatty substances preferably chosen from vegetable oils, liquid fatty esters of fatty acid and/or of fatty alcohol, amino silicones and mixtures thereof. More preferentially, they are chosen from sunflower, maize, soybean, marrow, grapeseed, sesame, hazelnut, apricot, macadamia, arara, castor, avocado oils, isopropyl myristate, coco-caprylate/caprate, amino silicones, such as amodimethicones, and bis-amino silicones, and mixtures thereof.

The total content of one or more fatty substances preferably varies from 0.1% to 30% by weight, preferentially from 0.5% to 20% by weight, more preferentially from 1% to 15% by weight, better still from 2% to 10% by weight, even more preferentially from 3% to 9% by weight, relative to the total weight of the composition.

The total content of one or more liquid fatty substances preferably varies from 0.1% to 30% by weight, preferentially from 0.5% to 20% by weight, more preferentially from 1% to 15% by weight, better still from 2% to 10% by weight, even more preferentially from 3% to 9% by weight, relative to the total weight of the composition.

### Cationic Polymer(s)

The solid composition according to the invention may also comprise one or more cationic polymers.

For the purposes of the present invention, the term “cationic polymer” means any polymer comprising cationic groups and/or groups that may be ionized into cationic groups. Preferably, the cationic polymer(s) are hydrophilic or amphiphilic.

The cationic polymers are preferably not silicone-based (they do not comprise any Si—O units).

The preferred cationic polymers are chosen from those that contain units including primary, secondary, tertiary and/or quaternary amine groups that may either form part of the main polymer chain or may be borne by a side substituent directly connected thereto.

Preferably, the cationic polymers according to the invention do not comprise any anionic groups or any groups that can be ionized into anionic groups.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:
(1) homopolymers or copolymers derived from acrylic or methacrylic esters or amides and including at least one of the units having the following formulae:

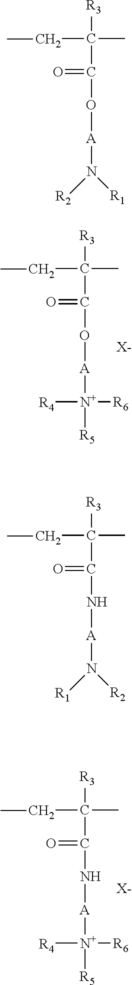

in which formulae:
   R₃, which may be identical or different, denote a hydrogen atom or a CH₃ radical;
   A, which may be identical or different, represent a linear or branched divalent alkyl group of 1 to 6 carbon atoms, preferably 2 or 3 carbon atoms, or a hydroxyalkyl group of 1 to 4 carbon atoms;
   R₄, R₅ and R₆, which may be identical or different, represent an alkyl group containing from 1 to 18 carbon atoms or a benzyl radical, and preferably an alkyl group containing from 1 to 6 carbon atoms;
   R₁ and R₂, which may be identical or different, represent a hydrogen atom or an alkyl group containing from 1 to 6 carbon atoms, preferably methyl or ethyl; and
   X denotes an anion derived from a mineral or organic acid, such as a methosulfate anion or a halide such as chloride or bromide.

The copolymers of the family (1) may also contain one or more units deriving from comonomers which may be chosen from the family of acrylamides, methacrylamides, diacetone acrylamides, acrylamides and methacrylamides substituted on the nitrogen with lower alkyls (C₁-C₄), acrylic acids or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, such as the products sold under the name Bina Quat P 100 by the company Ciba Geigy,
   the copolymer of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573,
   dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the products sold under the name Styleze CC 10 by ISP;
   quaternized vinylpyrrolidone/dimethylaminopropylmethacrylamide copolymers such as the product sold under the name Gafquat HS 100 by the company ISP;
polymers, preferably crosslinked polymers, of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.
(2) cationic polysaccharides, notably inulins, cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble quaternary ammonium monomer are described notably in patent U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by the company National Starch.

Among the cationic cellulose derivatives, use may also be made of cationic associative celluloses, which may be chosen from quaternized cellulose derivatives, and in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (VI):

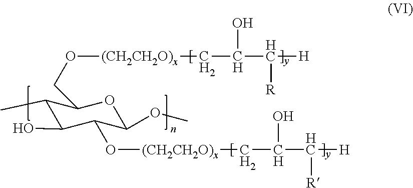

in which:
   R represents an ammonium group RaRbRcN⁺—, Q⁻ in which Ra, Rb and Rc, which may be identical or different, represent a hydrogen atom or a linear or branched C₁ to C₃₀ alkyl, preferably an alkyl, and Q⁻ represents an anionic counterion such as a halide, for instance a chloride or bromide;
   R′ represents an ammonium group R′aR′bR′cN⁺—, Q′⁻ in which R′a, R′b and R′c, which may be identical or different, represent a hydrogen atom or a linear or branched C₁ to C₃₀ alkyl, preferably an alkyl, and Q′⁻ represents an anionic counterion such as a halide, for instance a chloride or bromide;
it being understood that at least one of the radicals Ra, Rb, Rc, R′a, R′b and R′c represents a linear or branched C₈ to C₃₀ alkyl;
   n, x and y, which may be identical or different, represent an integer between 1 and 10 000.

Preferably, in formula (VI), at least one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radical(s) represent a linear or branched C₁-C₄ alkyl, notably methyl.

Preferably, in formula (VI), only one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radicals represent a linear or branched C₁ to C₄ alkyl, notably methyl.

Better still, R may be a group chosen from —N⁺(CH₃)₃, Q′⁻ and
—N⁺(C₁₂H₂₅)(CH₃)₂, Q′⁻, preferably a group —N⁺(CH₃)₃, Q′⁻.

Even better still, R′ may be a group —N*(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200®, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by the company Croda.

Mention may also be made of the hydroxyethylcelluloses of formula (VI) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from the company Amerchol/Dow Chemical.

More particularly, the polymers of formula (VI) are, for example, those whose viscosity is between 2 and 3 Pa·s (between 2000 and 3000 cPs) inclusive, preferentially between 2.7 and 2.8 Pa·s (between 2700 and 2800 cPs). Typically, Softcat Polymer SL-5 has a viscosity of 2.5 Pa·s (2500 cPs), Softcat Polymer SL-30 has a viscosity of 2700 cPs, Softcat Polymer SL-60 has a viscosity of 2.7 Pa·s (2700 cPs) and Softcat Polymer SL-100 has a viscosity of 2.8 Pa·s (2800 cPs). Use may also be made of Softcat Polymer SX-1300X with a viscosity of between 1 and 2 Pa·s (1000 and 2000 cPs.

The cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17 and Jaguar C162 by the company Rhodia.
(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.
(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they include one or more tertiary amine functions, they can be quaternized;
(5) polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or else under the name PD 170 or Delsette 101 by the company Hercules in the case of the adipic acid/epoxypropyl/diethylenetriamine copolymer.
(7) cyclopolymers of alkyldiallylamine or of dialkyldiallylammonium, such as homopolymers or copolymers including, as main constituent of the chain, units corresponding to formula (VII) or (VIII):

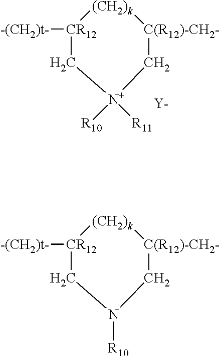

in which formulae (VII) and (VIII):
   k and t are equal to 0 or 1, the sum k+t being equal to 1;
   R₁₂ denotes a hydrogen atom or a methyl radical;
   R₁₀ and R₁₁, independently of each other, denote an alkyl group containing from 1 to 6 carbon atoms, a hydroxyalkyl group in which the alkyl group contains 1 to 5 carbon atoms, a C₁ to C4 amidoalkyl group; or alternatively R₁₀ and R₁₁ may denote, together with the nitrogen atom to which they are attached, heterocyclic groups such as piperidinyl or morpholinyl; R₁₀ and R₁₁, independently of each other, preferably denote an alkyl group containing from 1 to 4 carbon atoms; and
   Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold under the name Merquat 100 by the company Nalco (and homologues thereof of low weight-average molar masses) and the copolymers of diallyldimethylammonium salts (for example chloride) and of acrylamide, notably sold under the names Merquat 550 and Merquat 7SPR.
(8) quaternary diammonium polymers comprising repeating units of formula (IX):

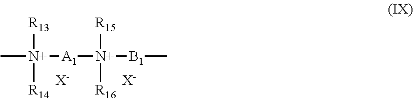

in which formula (IX):
   R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second non-nitrogen heteroatom, or alternatively R₁₃, R₁₄, R₁₅ and R₁₆ represent a linear or branched C₁ to C₆ alkyl radical substituted with a nitrile, ester, acyl or amide group or a group —CO—O—R₁₇-D or —CO—NH—R₁₇-D where R₁₇ is an alkylene and D is a quaternary ammonium group;
   A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
   X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B₁ can also denote a group (CH₂)ᵤCO-D-OC—(CH₂)ₙ— in which D denotes:
   a) a glycol residue of formula —O—Z—O—, in which Z denotes a linear or branched hydrocarbon-based radical or a group corresponding to one of the following formulae: —(CH₂—CH₂—O)ₓ—CH₂—CH₂— and —[CH₂CH(CH₃)—O]_{y}—CH₂—CH(CH₃)—, where x and y denote an integer from 1 to 4, representing a defined and unique degree of polymerization or any number from 1 to 4 representing an average degree of polymerization;
   b) a bis-secondary diamine residue, such as a piperazine derivative;
   c) a bis-primary diamine residue of formula: —NH—Y—NH—, where Y denotes a linear or branched hydrocarbon-based radical, or alternatively the divalent radical —CH₂—CH₂—S—S—CH₂—CH₂—; or
   d) a ureylene group of formula: —NH—CO—NH—.

Preferably, X⁻ is an anion, such as chloride or bromide. These polymers have a number-average molar mass (Mn) generally of between 1000 and 100 000.

Mention may be made more particularly of polymers consisting of repeating units corresponding to the formula (X):

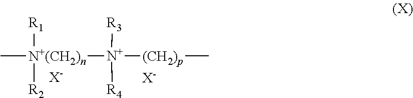

in which formula (X) R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (X) that is particularly preferred is the one for which R₁, R₂, R₃ and R₄ represent a methyl radical and n=3, p=6 and X═Cl, which is known as Hexadimethrine chloride according to the INCI (CTFA) nomenclature.
(9) polyquaternary ammonium polymers comprising units of formula (XI):

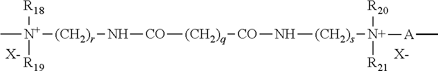

in which formula (XI):
   R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion, such as a halide, and
A denotes a dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by the company Miranol.
(10) quaternary polymers of vinylpyrrolidone and of vinylimidazole, for instance the products sold under the names Luviquat® FC 905, FC 550 and FC 370 by the company BASF.
(11) polyamines such as Polyquart® H sold by Cognis, which is referenced under the name Polyethylene Glycol (15) Tallow Polyamine in the CTFA dictionary.
(12) polymers including in their structure:
(a) one or more units corresponding to formula (A′) below:

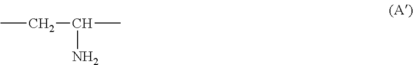

(b) optionally one or more units corresponding to formula (B′) below:

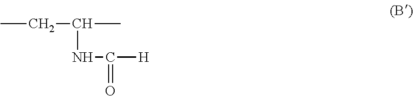

In other words, these polymers may be notably chosen from homopolymers or copolymers including one or more units derived from vinylamine and optionally one or more units derived from vinylformamide.

Preferably, these cationic polymers are chosen from polymers including, in their structure, from 5 mol % to 100 mol % of units corresponding to formula (A′) and from 0 to 95 mol % of units corresponding to formula (B′), preferentially from 10 mol % to 100 mol % of units corresponding to formula (A′) and from 0 to 90 mol % of units corresponding to formula (B′).

These polymers may be obtained, for example, by partial hydrolysis of polyvinylformamide. This hydrolysis may take place in acidic or basic medium.

The weight-average molecular mass of said polymer, measured by light scattering, may range from 1000 to 3 000 000 g/mol, preferably from 10 000 to 1 000 000 and more particularly from 100 000 to 500 000 g/mol.

The cationic charge density of these polymers may range from 2 meq/g to 20 meq/g, preferably from 2.5 to 15 meq/g and more particularly from 3.5 to 10 meq/g.

The polymers including units of formula (A) and optionally units of formula (B) are notably sold under the name Lupamin by the company BASF, for instance, in a non-limiting manner, the products sold under the names Lupamin 9095, Lupamin 5095, Lupamin 1095, Lupamin 9030 (or Luviquat 9030) and Lupamin 9010.

Preferably, the cationic polymer(s) are chosen from cationic polysaccharides (family (2)) and mixtures thereof, more preferentially from cationic galactomannan gums and mixtures thereof, and better still from cationic guar gums and mixtures thereof.

Preferably, the solid composition according to the invention comprises one or more cationic polymers.

More preferentially, the solid composition according to the invention comprises one or more cationic polymers chosen from cationic polysaccharides (family (2)) and mixtures thereof, more preferentially from cationic galactomannan gums and mixtures thereof and better still from cationic guar gums and mixtures thereof.

Preferentially again, the solid composition according to the invention comprises one or more cationic polymers chosen from cationic polysaccharides (family (2)), alkyldiallylamine or dialkyldiallylammonium cyclopolymers (family 7) and mixtures thereof, more preferentially still from mixtures of cationic galactomannan gums and of alkyldiallylamine or dialkyldiallylammonium cyclopolymers, better still from mixtures of cationic guar gums and copolymers of diallyldimethylammonium salts (for example chloride salt) and of acrylamide.

The total content of the cationic polymer(s) present in the solid composition according to the invention is preferably greater than or equal to 0.05% by weight, more preferentially ranges from 0.05% to 5% by weight, better still from 0.1% to 2% by weight and more preferentially still from 0.2% to 1.5% by weight, relative to the total weight of the composition.

According to a preferred embodiment, the cationic polymer(s) are chosen from cationic polysaccharides (family (2)) and mixtures thereof, and the total content of the cationic polysaccharide(s), present in the solid composition according to the invention, is preferably greater than or equal to 0.05% by weight, more preferentially ranges from 0.05% to 5% by weight and better still from 0.1% to 2% by weight, indeed even from 0.2% to 1.5% by weight, relative to the total weight of the composition.

### Polyol(s)

The solid composition according to the invention can additionally comprise one or more polyols.

The polyol(s) present in the solid composition of the invention are preferably chosen from the polyols of following formula (XII):

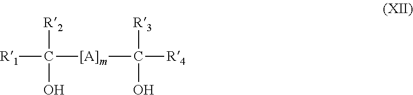

in which formula (XII):
   R′₁, R′₂, R′₃ and R′₄, which may be identical or different, denote, independently of one another, a hydrogen atom, a linear or branched C₁ to C₆ alkyl radical or a C₁ to C₆ mono- or polyhydroxyalkyl radical,
   A denotes a saturated or unsaturated, linear or branched, alkyl radical containing from 1 to 18 carbon atoms, this radical comprising from 0 to 9 oxygen atoms but no hydroxyl group, and
   m denotes 0 or 1.

The polyol(s) are preferably chosen from the polyols of formula (XII) in which m has the value 0, and mixtures thereof, and more preferentially from propylene glycol (propane-1,2-diol), 1,2,3-propanetriol, pinacol (2,3-dimethyl-2,3-butanediol), 1,2,3-butanetriol, 2,3-butanediol, glycerol, sorbitol and mixtures thereof.

The polyol(s) can also be chosen from the polyols of formula (XII) in which m has the value 1 and R′1, R′2, R′3 and R′4, which may be identical or different, denote, independently of one another, a hydrogen atom or a C₁ to C₆ alkyl radical, and mixtures thereof. They can advantageously be chosen from polyethylene glycols and mixtures thereof and more particularly the product referred to as PEG-6 or PEG-8 in the CTFA publication (International Cosmetic Ingredient Dictionary, Seven Edition).

The polyol(s) can also be chosen from the polyols of formula (XII) in which m has the value 1 and R′1, R′2, R′3 and R′4, which may be identical or different, denote, independently of one another, a hydrogen atom or a C1 to C6 alkyl radical, and the molecular weight of which is less than 200, and mixtures thereof. According to this particular embodiment, the polyol(s) are preferably chosen from 3-methyl-1,3,5-pentanetriol, 1,2,4-butanetriol, 1,5-pentanediol, 2-methyl-1,3-propanediol, 1,3-butanediol, 3-methyl-1,5-pentanediol, neopentyl glycol (2,2-dimethyl-1,3-propanediol), isoprene glycol (3-methyl-1,3-butanediol), hexylene glycol (2-methyl-2,4-pentanediol) and mixtures thereof, and more preferentially from hexylene glycol, neopentyl glycol, 3-methyl-1,5-pentanediol and mixtures thereof.

Preferably, the molecular weight (MW) of said polyol(s) present in the solid composition of the invention is between 50 and 350, more preferentially between 60 and 200 and better still between 70 and 150.

Preferably, the polyol(s) are chosen from diols, glycerol and mixtures thereof, more preferentially from the compounds of formula (XII) in which R′1, R′2, R′3 and R′4, which may be identical or different, denote, independently of one another, a hydrogen atom or a C1 to C6 alkyl radical, glycerol and mixtures thereof.

Advantageously, the polyol(s) are chosen from glycerol, propylene glycol (propane-1,2-diol), pinacol (2,3-dimethyl-2,3-butanediol), 2,3-butanediol, polyethylene glycols, 1,5-pentanediol, 2-methyl-1,3-propanediol, 1,3-butanediol, 3-methyl-1,5-pentanediol, neopentyl glycol (2,2-dimethyl-1,3-propanediol), isoprene glycol (3-methyl-1,3-butanediol), hexylene glycol (2-methyl-2,4-pentanediol), dipropylene glycol and mixtures thereof. Preferably, the polyol is glycerol or propylene glycol.

Preferably, the composition according to the invention comprises one or more polyols.

The total content of the polyol(s), present in the solid composition according to the invention, preferably ranges from 0.1% to 15% by weight, more preferentially from 0.5% to 10% by weight and better still from 1% to 5% by weight, relative to the total weight of the composition.

### Anticaking Agent(s)

The solid composition according to the invention can additionally comprise one or more anticaking agents.

For the purposes of the present invention, the term “anticaking agent” is intended to mean a lubricant acting as anticaking agent.

The lubricant(s) which can be used are different from the cationic polymers defined previously.

Mention may notably be made, among the lubricants which can be used in the solid composition of the invention, of silica, in particular anhydrous colloidal silica, sericite, polyamide (Nylon®), poly-p-alanine and polyethylene powders, tetrafluoroethylene (Teflon®) polymer powders, acrylate and dimethicone copolymers, stearic acid, metal soaps derived from organic carboxylic acids having from 8 to 22 carbon atoms, preferably from 12 to 18 carbon atoms, such as, for example, zinc, magnesium or lithium stearate, zinc laurate and magnesium myristate, alkali metal or alkaline-earth metal carbonates, such as, for example, magnesium, sodium and calcium carbonates, fatty acids, such as stearic acid, celluloses, notably crystalline celluloses, and mixtures thereof.

According to one embodiment, the anticaking agent(s) are advantageously chosen from metal soaps derived from organic carboxylic acids having from 8 to 22 carbon atoms, preferably from 12 to 18 carbon atoms, and mixtures thereof, better still from zinc stearate, magnesium stearate, lithium stearate, zinc laurate, magnesium myristate and mixtures thereof. More preferentially, the lubricant is magnesium stearate.

According to another embodiment, the anticaking agent(s) are advantageously chosen from alkali metal or alkaline-earth metal carbonates and mixtures thereof, preferably from magnesium carbonate, sodium carbonate, calcium carbonate and mixtures thereof; and more preferentially magnesium carbonate.

Preferably, the composition according to the invention comprises one or more anticaking agents.

The total content of the anticaking agent(s) present in the solid composition according to the invention preferably ranges from 0.1% to 20% by weight, more preferentially from 1% to 15% by weight and better still from 5% to 10% by weight, relative to the total weight of the composition.

### Carboxylic Acid(s)

The solid composition according to the present invention can comprise at least 10% by weight also of one or more C₁₋₆ carboxylic acids.

The carboxylic acid(s) preferably correspond(s) to the following formula (XIII):

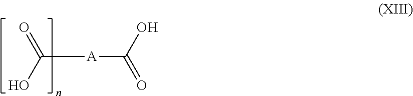

in which:
A represents a monovalent group when n has the value 0 or a polyvalent group when n is greater than or equal to 1; A represents a saturated or unsaturated, cyclic or non-cyclic, aromatic or non-aromatic, hydrocarbon-based group comprising from 1 to 6 carbon atoms, optionally interrupted by one or more heteroatoms and/or substituted by one or more hydroxyl and/or amino groups; preferably, A represents a monovalent C1-C6 alkyl or phenyl group or a polyvalent C1-C6 alkylene or phenylene group optionally substituted by one or more hydroxyl groups;
n represents an integer ranging from 0 to 10, preferably from 0 to 5, better still from 0 to 2.

More particularly, the carboxylic acid(s) of formula (XIII) are chosen from α-hydroxy acids, in which A represents a phenyl group, or a C1-C6, in particular C2-C4, alkylene group substituted by one or more hydroxyl groups; preferably by one OH group; and n has a value from 0 to 2.

More particularly, the C1-6 carboxylic acid(s) is or are chosen from those of formula (XIII) in which:
n=0 and A represents a C1-C6, in particular C2-C4, alkyl group, or a C1-C6, in particular C2-C4, alkyl group substituted by an OH group; or
n=0 and A represents a phenyl group, or a phenyl group substituted by an OH group; or
n=1 or 2 and A represents a di- or trivalent C1-C6, in particular C2-C4, alkyl group, or a di- or trivalent C₁-C₆, in particular C2-C4, alkyl group substituted by an OH group.

More preferentially still, the C1-6 carboxylic acid(s) is or are chosen from salicylic acid, citric acid, glutaric acid and lactic acid, and better still it is citric acid.

Preferably, the composition according to the invention comprises one or more C1-6 carboxylic acids.

When the C1-6 carboxylic acid(s) is or are present in the solid composition according to the invention, their total content preferably ranges from 10% to 30% by weight, preferentially from 11% to 25% by weight and better still from 12% to 20% by weight, relative to the total weight of the composition.

The solid composition according to the invention may also comprise water.

Preferably, the content of water is less than or equal to 20% by weight, preferentially less than or equal to 15% by weight, better still less than 10% by weight, relative to the total weight of the composition.

Preferably, the content of water ranges from 0.1% to 20% by weight, more preferentially from 1% to 15% by weight, and better still from 2% to 10% by weight, relative to the total weight of the composition.

The solid composition according to the invention can additionally comprise sodium chloride. Its content can range from 0.01% to 5% by weight, more preferentially from 0.1% to 3% by weight and better still from 0.2% to 2% by weight, relative to the total weight of the composition.

A subject of the present invention is also a process for the cosmetic treatment, notably for the care, of keratin fibres, in particular human keratin fibres such as the hair, comprising the application to said keratin fibres of a solid composition as defined above, the solid composition being applied directly to said keratin fibres or after having been moistened beforehand with water.

The solid composition according to the invention may be applied to dry or wet keratin fibres, preferably to wet keratin fibres.

The solid composition thus applied may optionally be rinsed off or left on, after an optional leave-on time that may range from 1 to 15 minutes, preferably from 2 to 10 minutes.

Preferably, the solid composition is rinsed off after application.

According to a first embodiment of the invention, the solid composition is applied directly to the keratin fibres, i.e. without being moistened and/or broken down in water beforehand.

When, according to this first embodiment, the solid composition of the invention is applied directly (i.e. without being moistened or broken down beforehand) to the dry keratin fibres, water may optionally be added to said fibres in order subsequently to rub/massage so as to dissolve said composition and to form a liquid composition. The liquid composition thus obtained can subsequently be rinsed out after an optional leave-on time.

Conversely, the solid composition of the invention may also be applied directly (i.e. without moistening or breaking down beforehand) to the wet keratin fibres, followed by massaging/rubbing to break down the particles and to obtain a liquid composition. The liquid composition thus obtained can subsequently be rinsed out after an optional leave-on time.

According to another embodiment of the invention, the solid composition is moistened and/or broken down beforehand in water before being applied to the keratin fibres. According to this embodiment, a small amount (preferably ranging from 1 to 3 g) of solid composition is advantageously taken up and dissolved with water, for example in the hand, so as to form a liquid composition. The liquid composition thus obtained may then be applied to the wet or dry keratin fibres, before being optionally rinsed out with water after an optional leave-on time.

The present invention also relates to the use of a solid composition as defined above for the cosmetic treatment, preferably the care, of keratin fibres, in particular human keratin fibres such as the hair.

The present invention also relates to a packaging article, preferably a cosmetic packaging article, comprising:
an envelope defining at least one cavity, the envelope comprising one or more water-soluble and/or liposoluble compounds;
a solid composition comprising: i) one or more cationic surfactants, ii) one or more starches, iii) one or more amphoteric surfactants, iv) one or more fatty substances;
it being understood that the solid composition is in one of the cavities defined by the envelope.

In the solid composition defined in the packaging article, said one or more cationic surfactants, said one or more starches, said one or more amphoteric surfactants and iv) said one or more fatty substances are as defined above.

Preferably, the solid composition in said packaging article comprises one or amphoteric surfactants in a total content ranging from 0.01% to 10% by weight, more preferentially from 0.05% to 5% by weight, and even better still from 0.1% to 1% by weight, relative to the total weight of the composition.

Furthermore, the solid composition in the packaging article is as defined above.

The solid composition in the packaging article may also comprise one or more cationic polymers as defined above.

The solid composition in the packaging article may also comprise one or more polyols as defined above.

The solid composition in the packaging article may also comprise one or more anticaking agents as defined above.

The solid composition in the packaging article may also comprise one or more one or more C₁₋₆ carboxylic acids as defined above.

The term “cosmetic packaging article” means an article that is suitable for cosmetic use; in particular for use of the packaging article on keratin fibres, notably the hair, and/or on the scalp. In particular, the packaging article makes it possible to condition the keratin fibres, in particular human keratin fibres such as the hair.

Preferably, the packaging article according to the invention is water-soluble or liposoluble at a temperature of less than or equal to 35 °C.

Preferably, the envelope of the packaging article according to the invention is water-soluble at a temperature of less than or equal to 35 °C.

The term “water-soluble” means soluble in water, in particular in a proportion of at least 10 grams per liter of water, preferably at least 20 g/1l, better still at least 50 g/l, at a temperature of less than or equal to 35 °C. Thus, when water preferably having a temperature of less than or equal to 35 °C is added to the packaging article, the envelope dissolves and releases the solid composition present in one of the cavities of the envelope.

The term “liposoluble” means soluble in a liquid fatty substance as defined below, in particular in a proportion of at least 10 grams per liter of liquid fatty substance, in particular in a plant or mineral oil such as liquid petroleum jelly, preferably at least 20 g/l in a liquid fatty substance, better still at least 50 g/l in a fatty substance, at a temperature of less than or equal to 35 °C.

The term “temperature of less than or equal to 35 °C” means a temperature not exceeding 35 °C but greater than or equal to 0 °C, for example ranging from more than 1 to 35 °C, preferably from 5 to 30 °C, more preferentially from 10 to 30 °C and better still from 15 to 25 °C. It is understood that all the temperatures are given at atmospheric pressure (1 atm).

The packaging article may comprise one or more cavities, at least one of which contains the solid composition as defined previously. Preferably, the packaging article comprises only one cavity in which the solid composition is contained.

Advantageously, the envelope represents from 0.5% to 20% by weight, preferably from 1% to 15% by weight, more preferentially from 2% to 10% by weight and better still from 4% to 8% by weight relative to the total weight of the packaging article.

Advantageously, the solid composition as defined previously represents from 80% to 99.5% by weight, preferably from 85% to 99% by weight, more preferentially from 90% to 98% by weight and better still from 92% to 96% by weight relative to the total weight of the packaging article.

The weight ratio between the total weight of the solid composition of the invention and the total weight of the envelope advantageously ranges from 80/20 to 99/1, preferably from 85/15 to 98/2 and more preferentially from 90/10 to 97/3.

The envelope of the packaging article comprises one or more water-soluble and/or liposoluble compounds, preferably one or more water-soluble compounds advantageously chosen from water-soluble polymers and mixtures thereof.

The water-soluble polymer(s) that may be used according to the present invention contain water-soluble units in their backbones. The water-soluble units are obtained from one or more water-soluble monomers.

The term “water-soluble monomer” means a monomer whose solubility in water is greater than or equal to 1%, preferably greater than or equal to 5%, at 25 °C and at atmospheric pressure (760 mmHg).

Said water-soluble polymer(s) that are capable of forming the envelope are advantageously obtained from water-soluble monomers including at least one double bond. These monomers may be chosen from cationic, anionic and nonionic monomers, and mixtures thereof.

As water-soluble monomers that may be used as precursors for the water-soluble units, alone or as a mixture, examples that may be mentioned include the following monomers, which may be in free or salified form:
(meth)acrylic acid,
styrenesulfonic acid,
vinylsulfonic acid and (meth)allylsulfonic acid,
vinylphosphonic acid,
N-vinylacetamide and N-methyl-N-vinylacetamide,
N-vinylformamide and N-methyl-N-vinylformamide,
N-vinyllactams including a cyclic alkyl group containing from 4 to 9 carbon atoms, such as N-vinylpyrrolidone, N-butyrolactam and N-vinylcaprolactam,
maleic anhydride,
itaconic acid,
vinyl alcohol of formula CH₂═CHOH,
vinyl acetate of formula CH₂═CHOC(O)CH₃,
vinyl ethers of formula CH₂═CHOR in which R is a linear or branched, saturated or unsaturated hydrocarbon-based radical containing from 1 to 6 carbon atoms;
dimethyldiallylammonium halides (chloride),
quaternized dimethylaminomethyl methacrylate (DMAEMA),
(meth)acrylamidopropyltrimethylammonium halides (chloride) (APTAC and MAPTAC),
methylvinylimidazolium halides (chloride),
2-vinylpyridine and 4-vinylpyridine,
acrylonitrile,
glycidyl (meth)acrylate,
vinyl halides (chloride) and vinylidene chloride,
the vinyl monomers having the following formula: H₂C═C(R)—C(O)—X, in which:
   R is chosen from H, (C₁-C₆)alkyl such as methyl, ethyl and propyl, and
   X is chosen from:
      alkoxy groups of the type —OR′ in which R′ is a linear or branched, saturated or unsaturated hydrocarbon-based radical containing from 1 to 6 carbons, optionally substituted with at least one halogen (iodine, bromine, chlorine or fluorine); a group from among sulfonic (—SO₃⁻), sulfate (SO₄⁻), phosphate (—PO₄H₂); hydroxyl (—OH); primary amine (—NH₂); secondary amine (NHR₆), tertiary amine (—NR₆R₇) or quaternary amine (—N⁺R₆R₇R₈) with R₆, R₇ and R₈ being, independently of each other, a linear or branched, saturated or unsaturated hydrocarbon-based radical containing 1 to 6 carbon atoms, with the proviso that the sum of the carbon atoms of R′+R₆+R₇+R₈ does not exceed 6;
      groups —NH₂, —NHR′ and —NR′R″ in which R′ and R″ are, independently of each other, linear or branched, saturated or unsaturated hydrocarbon-based radicals containing from 1 to 6 carbons, with the proviso that the total number of carbon atoms of R′+R″ does not exceed 6, said radicals R′ and R″ being optionally substituted with a halogen (iodine, bromine, chlorine or fluorine); a group from among hydroxyl (—OH); sulfonic (—SO₃₋), sulfate (SO₄⁻), phosphate (—PO₄H₂); primary amine (—NH₂); secondary amine (NHR₆), tertiary amine (—NR₆R₇) and/or quaternary amine (—N⁺R₆R₇R₈) with R₆, R₇ and R₈ being, independently of each other, a linear or branched, saturated or unsaturated hydrocarbon-based radical containing 1 to 6 carbon atoms, with the proviso that the sum of the carbon atoms of R′+R″+R₆+R₇+R₈ does not exceed 6. As compounds corresponding to this formula, examples that may be mentioned include N,N-dimethylacrylamide and N,N-diethylacrylamide;
   and mixtures thereof.

Anionic monomers that may notably be mentioned include (meth)acrylic acid, acrylamido-2-methylpropanesulfonic acid, itaconic acid and the salts thereof with an alkali metal, an alkaline-earth metal or ammonium or those derived from an organic amine such as an alkanolamine.

Nonionic monomers that may notably be mentioned include (meth)acrylamide, N-vinylformamide, N-vinylacetamide and hydroxypropyl (meth)acrylate, vinyl alcohol of formula CH₂═CHOH, and vinyl acetate of formula CH₂═CHOC(O)CH₃.

The cationic monomers are preferably chosen from quaternary ammonium salts derived from a diallylamine, and those corresponding to the following formula:

H₂C═C(R₁)-D-N⁺R₂R₃R₄,X⁻
in which:
   R₁ represents a hydrogen atom or a methyl group,
   R₂ and R₃, which may be identical or different, represent a hydrogen atom or a linear or branched C₁ to C₄ alkyl group,
   R₄ represents a hydrogen atom, a linear or branched C₁-C₄ alkyl group or an aryl group,
   D represents the following divalent unit: —(Y)ₙ-(A)- in which:
      Y represents an amide function, an ester (O—C(O) or C(O)—O), a urethane or a urea,
      A represents a linear or branched, cyclic or acyclic C₁ to C₁₀ alkylene group, which may be substituted or interrupted with a divalent aromatic or heteroaromatic group. The alkylene groups may be interrupted with an oxygen atom, a nitrogen atom, a sulfur atom or a phosphorus atom; the alkylene possibly being interrupted with a ketone function, an amide, an ester (O—C(O) or C(O)—O), a urethane or a urea,
   n is an integer ranging from 0 to 1,
X⁻ represents an anionic counterion, for instance a chloride or a sulfate.

Examples of water-soluble cationic monomers that may notably be mentioned include the following compounds, and also the salts thereof: dimethylaminoethyl (meth)acrylate, (meth)acryloyloxyethyltrimethylammonium (meth)acrylate, (meth)acryloyloxyethyldimethylbenzylammonium (meth)acrylate, N-[dimethylaminopropyl](meth)acrylamide (meth)acrylate, (meth)acrylamidopropyltrimethylammonium (meth)acrylate, (meth)acrylamidopropyldimethylbenzylammonium (meth)acrylate, dimethylaminohydroxypropyl (meth)acrylate, (meth)acryloyloxyhydroxypropyltrimethylammonium (meth)acrylate, (meth)acryloyloxyhydroxypropyldimethylbenzylammonium (meth)acrylate and dimethyldiallylammonium (meth)acrylate.

Among the water-soluble polymers that may be used according to the present invention, mention may also be made of polyhydroxyalcanoates (PHA), poly(hydroxybutyrate)s (PHB), poly(hydroxyvalerate)s (PHV), biodegradable polyesters and mixtures thereof.

Preferably, the water-soluble polymers are polymerized from one or more monomers chosen from vinyl alcohol of formula CH₂═CHOH, vinyl acetate of formula CH₂═CHOC(O)CH₃ and mixtures thereof.

The water-soluble polymers that are capable of forming the envelope of the packaging article may also be chosen from water-soluble polymers derived from natural products, such as polysaccharides, i.e. polymers bearing sugar units. These water-soluble polymers are different from the cationic polysaccharide(s) which may be present in the solid composition.

The term “sugar unit” means a unit derived from a carbohydrate of formula Cₙ(H₂O)ₙ₋₁ or (CH₂O)ₙ, which may be optionally modified by substitution and/or by oxidation and/or by dehydration. The sugar units that may be included in the composition of the polymers of the invention are preferably derived from the following sugars: glucose, galactose, arabinose, rhamnose, mannose, xylose, fucose, fructose, anhydrogalactose, galacturonic acid, glucuronic acid, mannuronic acid, galactose sulfate, anhydrogalactose sulfate.

The polymers bearing sugar unit(s) according to the invention may be of natural or synthetic origin. They may be nonionic, anionic, cationic or amphoteric. The base units of the polymers bearing a sugar unit of the invention may be monosaccharides or disaccharides.

As polymers that may be used, mention may notably be made of the following native gums, and also derivatives thereof:
a) tree or shrub exudates, including:
acacia gum (branched polymer of galactose, arabinose, rhamnose and glucuronic acid);
ghatti gum (polymer derived from arabinose, galactose, mannose, xylose and glucuronic acid);
karaya gum (polymer derived from galacturonic acid, galactose, rhamnose and glucuronic acid);
gum tragacanth (or tragacanth) (polymer of galacturonic acid, galactose, fucose, xylose and arabinose);
b) gums derived from algae, including:
agar (polymer derived from galactose and anhydrogalactose);
alginates (polymers of mannuronic acid and of glucuronic acid);
carrageenans and furcellerans (polymers of galactose sulfate and of anhydrogalactose sulfate);
c) gums derived from seeds or tubers, including:
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose), the main constituent of which is glucomannan, which is a polysaccharide of high molecular weight (500 000<M_{glucomannan}<2 000 000) composed of D-mannose and D-glucose units with a branch every 50 or 60 units approximately;
d) microbial gums, including:
xanthan gum (polymer of glucose, mannose acetate, mannose/pyruvic acid and glucuronic acid);
gellan gum (polymer of partially acylated glucose, rhamnose and glucuronic acid);
scleroglucan gum (glucose polymer);
biosaccharide gum (polymer of galacturonic acid, fucose and D-galactose),
e) plant extracts, including:
cellulose (glucose polymer);
natural starch (glucose polymer) or modified starch (aluminium starch octenylsuccinate, hydroxypropyl starch phosphates);
inulin (polymer of fructose and glucose).

These polymers may be physically or chemically modified. A physical treatment that may notably be mentioned is the temperature. Chemical treatments that may be mentioned include esterification, etherification, amidation and oxidation reactions. These treatments can lead to polymers that may be nonionic, anionic, cationic or amphoteric.

Preferably, these chemical or physical treatments are applied to guar gums, locust bean gums, starches and celluloses.

The nonionic guar gums that may be used according to the invention may be modified with C₁ to C₆ hydroxyalkyl groups. Among the hydroxyalkyl groups, mention may be made of hydroxymethyl, hydroxyethyl, hydroxypropyl and hydroxybutyl groups.

These guar gums are well known in the prior art and may be prepared, for example, by reacting corresponding alkene oxides, for instance propylene oxides, with the guar gum so as to obtain a guar gum modified with hydroxypropyl groups.

The degree of hydroxyalkylation preferably ranges from 0.4 to 1.2 and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The guar gums modified with cationic groups that may more particularly be used according to the invention are guar gums including trialkylammonium cationic groups. Preferably, 2% to 30% by number of the hydroxyl functions of these guar gums bear trialkylammonium cationic groups. Even more preferentially, 5% to 20% by number of the hydroxyl functions of these guar gums are branched with trialkylammonium cationic groups. Among these trialkylammonium groups, mention may most particularly be made of the trimethylammonium and triethylammonium groups. Even more preferentially, these groups represent from 5% to 20% by weight relative to the total weight of the modified guar gum.

According to the invention, guar gums modified with 2,3-epoxypropyltrimethylammonium chloride may be used.

These guar gums modified with cationic groups are products already known per se and are, for example, described in patents U.S. Pat. Nos. 3,589,578 and 4,013,307. Such products are moreover notably sold under the trade names Jaguar C13S, Jaguar C15 and Jaguar C17 by the company Rhodia Chimie.

As modified locust bean gum, use may be made of cationic locust bean gum containing hydroxypropyltrimonium groups, such as Catinal CLB 200 sold by the company Toho.

Starch molecules which may be used in the envelope of the packaging article according to the present invention are the same as those described above for the solid composition.

Preferably, starches of formula (VIIa) or (VIIIa), and preferentially starches modified with 2-chloroethylaminodipropionic acid will particularly be used, i.e. starches of formula (VIIa) or (VIIIa) in which R, R′, R″ and M represent a hydrogen atom and n is equal to 2. Preferably, the amphoteric starch is a starch chloroethylamido dipropionate.

The celluloses and cellulose derivatives may be anionic, cationic, amphoteric or nonionic.

Among these derivatives, cellulose ethers, cellulose esters and cellulose ester ethers are distinguished.

Among the cellulose esters, mention may be made of inorganic esters of cellulose (cellulose nitrates, sulfates or phosphates), organic esters of cellulose (cellulose monoacetates, triacetates, amidopropionates, acetatebutyrates, acetatepropionates or acetatetrimellitates), and mixed organic/inorganic esters of cellulose, such as cellulose acetatebutyrate sulfates and cellulose acetatepropionate sulfates.

Among the cellulose ester ethers, mention may be made of hydroxypropylmethylcellulose phthalates and ethylcellulose sulfates.

Among the nonionic cellulose ethers that may be mentioned are alkylcelluloses such as methylcelluloses and ethylcelluloses (for example Ethocel Standard 100 Premium from Dow Chemical); hydroxyalkylcelluloses such as hydroxymethylcelluloses and hydroxyethylcelluloses (for example Natrosol 250 HHR sold by Aqualon) and hydroxypropylcelluloses (for example Klucel EF from Aqualon); mixed hydroxyalkyl-alkylcelluloses such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Among the anionic cellulose ethers, mention may be made of carboxyalkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and also the sodium salts thereof.

Among the cationic cellulose ethers, mention may be made of crosslinked or non-crosslinked quaternized hydroxyethylcelluloses. The quaternizing agent may notably be diallyldimethylammonium chloride (for example Celquat L200 from National Starch). Another cationic cellulose ether that may be mentioned is hydroxypropyltrimethylammonium hydroxyethyl cellulose (for example Ucare Polymer JR 400 from Amerchol).

Among the associative polymers bearing sugar unit(s), mention may be made of celluloses or derivatives thereof, modified with groups including at least one fatty chain such as alkyl, arylalkyl or alkylaryl groups or mixtures thereof, in which the alkyl groups are C8-C22; nonionic alkylhydroxyethylcelluloses such as the products Natrosol Plus Grade 330 CS and Polysurf 67 (C₁₆ alkyl) sold by the company Aqualon; quaternized alkylhydroxyethylcelluloses (cationic) such as the products Quatrisoft LM 200, Quatrisoft LM-X 529-18-A, Quatrisoft LM-X5 29-18-B (C₁₂ alkyl) and Quatrisoft LM-X 529-8 (C₁₈ alkyl) sold by the company Amerchol, the products Crodacel QM, Crodacel QL (C₁₂ alkyl) and Crodacel QS (C₁₈ alkyl) sold by the company Croda and the product Softcat SL 100 sold by the company Amerchol; nonionic nonoxynylhydroxyethylcelluloses such as the product Amercell HM-1500 sold by the company Amerchol; nonionic alkylcelluloses such as the product Bermocoll EHM 100 sold by the company Berol Nobel.

As associative polymers bearing sugar unit(s) derived from guar, mention may be made of hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

The water-soluble polymer(s) bearing sugar unit(s) that may be used to form the envelope of the packaging article are preferably chosen from guar gums, locust bean gums, xanthan gums, starches and celluloses, in their modified (derived) form or unmodified form.

Preferably, said polymer(s) bearing sugar unit(s) are nonionic.

The water-soluble polymers described above more particularly have a weight-average molecular weight (Mw) of greater than 1 000 000 and preferably between 1 000000 and 50 000 000. The molecular weight is determined by the RSV (Reduced Specific Viscosity) method as defined in “Principles of Polymer Chemistry” Cornell University Press, Ithaca, NY 1953 Chapter VII “Determination of Molecular Weight” pages 266-316.

The water-soluble or liposoluble compound(s) that are capable of forming the envelope of the packaging article according to the invention may be in fibre or film form.

According to a first embodiment, the water-soluble or liposoluble compound(s) are in the form of fibres. The term “fibre” refers to any object whose length is greater than its cross section. In other words, it should be understood as referring to an object of length L and of diameter D such that L is greater and preferably very much greater (i.e. at least three times greater) than D, D being the diameter of the circle in which the cross section of the fibre is inscribed. In particular, the ratio L/D (or aspect ratio) is chosen in the range extending from 3.5 to 2500, preferably from 5 to 500, and better still from 5 to 150. The cross section of a fibre may be of any shape: round, serrated or crenellated, or else bean-shaped, but also multilobal, in particular trilobal or pentalobal, X-shaped, in strip form, square, triangular, elliptical or the like. The fibres of the invention may or may not be hollow.

According to this embodiment, the fibres may be spun, carded or twisted. Advantageously, the fibres used in the context of the present invention are spun. The mean diameter of the fibres used according to the present invention, which may be identical or different, is less than 500 μm. Advantageously, such a diameter is less than 200 μm, preferably less than 100 μm, or even less than 50 μm.

Mention may be made more particularly of water-soluble fibres which include fibres based on poly(vinyl alcohol) (=PVA), fibres of polysaccharides such as glucomannans, starches, celluloses such as carboxymethylcelluloses, polyalginic acid fibres, polylactic acid fibres and polyalkylene oxide fibres, and also mixtures thereof. More preferentially, the water-soluble fibre(s) used in the invention are chosen from PVA-based fibres.

The fibres of the envelope are generally entangled. The term “envelope comprising water-soluble fibres” means an envelope which may consist entirely of water-soluble fibres which may include both fibres that are water-soluble and fibres that are water-insoluble at a temperature of less than or equal to 35 °C, the soluble fibres needing to be in larger amount than the insoluble fibres. The envelope of the fibres must include at least 60% by weight of soluble fibres, preferably at least 70% and better still at least 80% by weight relative to the total weight of the fibres. It may thus include, for example, more than 95% by weight, or even more than 99% by weight and even 100% by weight of water-soluble fibres relative to the total weight of the fibres of the envelope.

When the envelope contains insoluble fibres, these may be made of any material commonly used as insoluble fibres; they may be, for example, silk, cotton, wool, flax, polyamide (Nylon®), polylactic acid, modified cellulose (rayon, viscose, rayon acetate), poly-p-phenylene terephthalamide, notably Kevlar®, polyolefin and notably polyethylene or polypropylene, glass, silica, aramid, carbon, notably in graphite form, Teflon®, insoluble collagen, polyester, polyvinyl chloride or polyvinylidene chloride or polyethylene terephthalate fibres, or fibres formed from a mixture of the compounds mentioned above, such as polyamide/polyester or viscose/polyester fibres.

In addition, when the envelope contains fibres, it may be woven or nonwoven.

According to a first variant of the invention, the envelope may be woven. In the context of the present invention, a “woven” material results from an organized assembly of fibres, in particular of water-soluble polymeric fibres, and more particularly of an intercrossing, in the same plane, of said fibres, arranged in the direction of the warp and of fibres arranged, perpendicular to the warp fibres, in the direction of the weft. The bonding obtained between these warp and weft fibres is defined by a weave.

Such a woven material results from an operation directed towards assembling the fibres in an organized manner such as weaving per se, but may also result from knitting.

According to another variant of the invention, the envelope is nonwoven.

For the purposes of the present invention, the term “nonwoven fabric” refers to a substrate comprising fibres, in particular water-soluble polymeric fibres, in which the individual fibres are arranged in a disordered manner in a structure in the form of a lap and which are neither woven nor knitted. The fibres of the nonwoven fabric are generally bonded together, either under the effect of a mechanical action (for example needle punching, air jet or water jet), or under the effect of a thermal action, or by addition of a binder.

Such a nonwoven fabric is, for example, defined by the standard ISO 9092 as a web or lap of directionally or randomly oriented fibres, bonded by friction and/or cohesion and/or adhesion, excluding paper and products which are woven, knitted, tufted or stitch-bonded incorporating bonding yarns or filaments.

A nonwoven fabric differs from a paper by the length of the fibres used. In paper, the fibres are shorter. However, there are nonwoven fabrics based on cellulose fibre, which are manufactured by a wet-laid process and which have short fibres like in paper. The difference between a nonwoven fabric and a paper is generally the absence of hydrogen bonding between the fibres in a nonwoven fabric.

Very preferentially, the fibres used in the context of the present invention are chosen from synthetic fibres such as PVA fibres. In particular, the envelope is nonwoven, and is preferentially made of nonwoven PVA fibres.

To make the nonwoven envelope of the packaging article, use is preferably made of PVA fibres that are soluble in water at a temperature of less than or equal to 35 °C, for instance the fibres sold by the Japanese company Kuraray under the name Kuralon K-II, and particularly the grade WN2 which is soluble at and above 20 °C. These fibres are described in EP-A-636 716 which teaches the manufacture of PVA fibres that are soluble in water at temperatures not exceeding 100 °C, by spinning and drawing of the wet or dry polyvinyl alcohol polymer in the presence of solvents participating in the dissolution and solidification of the fibre. The fibre thus obtained may lead to the production of woven or nonwoven substrates.

These fibres may also be prepared from a solution to be spun, by dissolving a water-soluble PVA-based polymer in a first organic solvent, spinning of the solution in a second organic solvent to obtain solidified filaments and wet drawing of the filaments, from which the first solvent is removed, followed by drying and subjecting to a heat treatment. The cross section of these fibres may be substantially circular. These fibres have a tensile strength of at least 2.7 g/dtex (3 g/d). Patent application EP-A-0 636 716 describes such water-soluble PVA-based fibres and the process for manufacturing them. For example, the fibres may also be formed by extrusion and deposited on a conveyor to form a lap of fibres which is then consolidated via a conventional fibre bonding technique, for instance needle punching, hot bonding, calendering or air-through bonding, in which technique the water-soluble lap passes through a tunnel into which hot air is blown, or spunlacing directed towards bonding the fibres under the action of fine jets of water at very high pressure, which cannot be applied to fibres whose dissolution temperature is too low.

As has been seen previously, the invention is not limited to the use of PVA, and use may also be made of fibres made from other water-soluble materials provided that these materials dissolve in water having the desired temperature, for example the polysaccharide fibres sold under the name Lysorb by the company Lysac Technologies Inc. or other fibres based on polysaccharide polymers such as glucomannans or starch.

The envelope may comprise a mixture of different fibres that are soluble in water at different temperatures (up to 35 °C).

The fibres may be composite, and they may include, for example, a core and a sheath which are not of the same nature, for example formed from different grades of PVA.

According to a particular embodiment of the invention, the envelope is a nonwoven fabric, including water-soluble fibres, alone or as a mixture with insoluble fibres as indicated above, with not more than 40% by weight of insoluble fibres relative to the total weight of the fibres constituting the lap. Preferably, the nonwoven fabric consists essentially of water-soluble fibres, i.e. it does not contain any insoluble fibres.

According to another embodiment of the invention, the envelope of the packaging article may consist of one or more films, which each comprise one or more water-soluble and/or liposoluble compounds, notably as defined above. When the envelope consists of several films, said films may be assembled, for example bonded together, so as to form a single unified film.

The thickness of the “overall” film (i.e. the thickness of the single film when the envelope contains only one or of the unified film when the envelope contains several films) is advantageously between 10 and 1000 microns, preferably between 10 and 800 microns and more preferentially between 15-500 microns.

The term “film” notably means a continuous layer preferentially formed from one or more water-soluble and/or liposoluble compounds as defined above, in particular of polymer(s).

The main industrial methods for the production of polymer films are extrusion of a molten polymer, casting of a solution of a polymer onto a polished metal surface (in certain cases, the polymer solution is introduced into a precipitation tank), casting of a dispersion of the polymer onto a polished surface, and calendering.

The films that may be used according to the present invention may be chosen from film-multilayer film, film-paper (laminating) and film-coating.

During application by spraying, brushing or various industrial processes, the surface coatings undergo what is known as the formation of a film, and notably of film-coating. In the majority of the film-forming processes, a liquid coating of relatively low viscosity is applied to a solid substrate and is hardened as a solid adherent film based on high molecular weight polymer having the properties desired by the user.

The films that may be used according to the present invention are notably PVA films which may be manufactured via any industrial production method, such as a method of casting a PVA-based polymer solution, a method of extrusion in the presence or absence of water, a dry-extrusion moulding method or a biaxial orientation method.

The packaging article, and the envelope, may have any shape that is suitable for the intended use, for example a rectangular, round or oval shape. Preferably, it has a rounded geometry, for example in the form of a sphere, a disc or an oval, or else a square or parallelepipedal geometry preferably with rounded corners. The envelope preferably has dimensions allowing it to be taken up between at least two fingers. Thus, it may, for example, have an ovoid shape about 2 to 10 cm long and about 0.5 to 4 cm wide, or a circular disc shape about 2 to 10 cm in diameter, or a square shape with a side length of about 2 to 15 cm, or a rectangular shape with a length of about 2 to 25 cm, it being understood that it may have any other shape and size that are suitable for the intended use.

Preferably, the envelope may be of round shape with an inside diameter ranging from 3 to 7 cm, more preferentially from 4 to 5 cm; to which may be added the dimension of the edges (sealed part) which may range from 1 to 5 mm, better still from 2 to 4 mm; and a height ranging from 2 to 7 mm, preferentially from 3 to 5 mm.

The envelope may also be of square or rectangular shape with a length preferably ranging from 2 to 6 cm, more preferentially from 3 to 5 cm, and a width preferably ranging from 2 to 5 cm, more preferentially 2.5 to 4 cm; to which may be added the dimension of the edges (sealed part) which may preferably range from 1 to 5 mm, and more preferentially from 2 to 4 mm.

Advantageously, the envelope has a low thickness, and may consist of several layers of different materials. Preferably, the thickness of the envelope ranges from 3% to 99.9% of its other dimensions. The envelope is thus substantially flat, with thin edge profiles.

The area delimiting the cavity or cavities has an extent advantageously less than 625 cm², preferably between 0.025 cm² and 400 cm², more preferentially between 1 and 200 cm², better still between 2 and 50 cm² and even better still between 4 and 25 cm², so as to have optimized compacting of the composition. It has been observed that when the area of the article is within the above ranges, the compacting of the solid composition made of powder is lower and the transformation of the powder into a fluid composition in the hands is easier, without any formation of agglomerates.

Preferably, the height of the envelope is greater than or equal to 2 mm, more preferentially ranging from 2 to 10 mm and better still from 3 to 7 mm.

Preferably, the film(s) used in the context of the present invention are chosen from synthetic films such as PVA films, and also mixtures thereof.

Preferably, the envelope consists of several layers, for example two or three layers, of films which are each preferably made of different materials. Advantageously, at least one of these films is a film comprising or consisting of PVA.

Preferably, the film(s) are sealed so as to form one or more cavities which will comprise the solid composition of the invention and will prevent it from escaping.

Advantageously, the envelope represents from 0.5% to 20% by weight, preferably from 1% to 15% by weight, more preferentially from 2% to 10% by weight, better still from 4% to 10% by weight, and even better still from 4% to 8% by weight relative to the total weight of the packaging article.

Advantageously, the solid composition as defined below represents from 80% to 99.5% by weight, preferably from 85% to 99% by weight, more preferentially from 90% to 98% by weight, better still from 90% to 96% by weight, and even better still from 92% to 96% by weight relative to the total weight of the packaging article.

The weight ratio between the total weight of the solid composition of the invention and the total weight of the envelope advantageously ranges from 80/20 to 99/1, preferably from 85/15 to 98/2 and more preferentially from 90/10 to 97/3.

Advantageously, the packaging article comprises from 1 to 8 g and preferably from 2 to 6 g of solid composition; and from 0.1 to 1 g and preferably from 0.2 to 0.7 g of envelope.

The present invention also relates to a cosmetic process for treating keratin fibres, in particular human keratin fibres such as the hair, comprising a step of using a packaging article as defined above; preferably, said cosmetic treatment process comprises the following steps:
i) mixing the packaging article in a composition that is capable of dissolving, totally or partially, the envelope of said packaging article,
ii) applying the composition obtained in step i) to the keratin fibres,
iii) optionally leaving to rest,
iv) rinsing said keratin fibres, and
v) optionally drying said keratin fibres.

It is understood that the composition that is suitable for dissolving the envelope depends on the nature of the envelope. In other words, the composition that is suitable for dissolving the envelope is water or an aqueous composition when the packaging article predominantly or solely contains a hydrophilic envelope. Further, the composition that is suitable for dissolving the envelope is an anhydrous organic composition or an aqueous composition comprising at least one liquid fatty substance or at least one organic solvent other than liquid fatty substances such as lower monoalcohols, for example ethanol, or such as polyols, for example propylene glycol or glycerol, when the packaging article predominantly or solely contains a lipophilic envelope.

Thus, the aqueous composition may simply be water. The aqueous composition may optionally comprise at least one polar solvent. Among the polar solvents that may be used in this composition, mention may be made of organic compounds that are liquid at room temperature (25 °C) and at least partially water-miscible.

Examples that may be mentioned more particularly include alcohols such as ethyl alcohol and isopropyl alcohol, aromatic alcohols such as benzyl alcohol and phenylethyl alcohol, or polyols or polyol ethers, for instance ethylene glycol monomethyl ether, monoethyl ether and monobutyl ether, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether.

More particularly, if one or more solvents are present, their respective content in the aqueous composition ranges from 0.5% to 20% by weight and preferably from 2% to 10% by weight relative to the weight of said aqueous composition.

The dilution ratio (expressed by weight) between one or more packaging articles, as defined previously, and the composition that is suitable for dissolving the packaging article(s) is preferably between 10/90 and 90/10 and more preferentially between 10/90 and 50/50. Better still, this dilution ratio is 20/80.

In particular, the composition obtained on conclusion of the mixing (step i) of the process) may be applied to wet or dry keratin fibres. It is advantageously left in place on the keratin fibres for a time generally ranging from 1 to 15 minutes, preferably from 2 to 10 minutes.

The keratin fibres are then rinsed with water. They may optionally be washed with a shampoo, followed by rinsing with water, before being dried or left to dry.

The present invention also relates to the use of a packaging article as defined previously for the cosmetic treatment, more particularly the care, of keratin fibres, in particular human keratin fibres such as the hair.

The examples that follow serve to illustrate the invention without, however, being limiting in nature.

### EXAMPLES

In the examples which follow, all the amounts are shown, unless otherwise indicated, as weight percentage of active material relative to the total weight of the composition.

### Example 1

A composition according to the invention was prepared from the ingredients indicated in Table 1 below:

TABLE 1
3

| | Composition 1 |
|---|---|
| Behentrimonium chloride | 2.4 |
| Maize starch (Zea mays (corn) starch) | 43.3 |
| Distarch phosphate | 10 |
| Citric acid | 14.8 |
| Soybean oil | 1 |
| Isopropyl myristate | 2 |
| Sunflower oil | 0.5 |
| Shea butter (Butyrospermium Parkii | 3 |
| Butter) | |
| Coco-caprylate/caprate | 0.5 |
| Cocamidopropylbetaine | 0.2 |
| Sodium chloride | 0.5 |
| Magnesium carbonate | 7 |
| Isopropyl alcohol | 0.5 |
| Hydroxypropyl guar | 0.8 |
| hydroxypropyltrimonium chloride | |
| Water | 7 |
| Glycerol | 3 |
| Fragrance | 3.5 |

The powder ingredients (maize starch, starch phosphate, citric acid, guar) were mixed with stirring in a vessel. The liquid ingredients (except for the fragrance) are heated to 40 °C. These ingredients are added as binder to the vessel by spraying and mixed. At the end, the fragrance is added by spraying and mixing is carried out. The combined mixture is subsequently dried in an oven at 45 °C for 20 hours.

After cooling to ambient temperature, the mixture is ground using a mill in order to obtain a powder. A fairly free powder formula (with a talus angle of 30°) and a density of 0.48 and a water activity of 0.628 (after drying) is obtained. Size of particles before grinding X50%=286 μm and after grinding X50%=216 μm.

In accordance with the present invention, the talus angle represents the angle characteristic of a pile of powder obtained by mechanically pouring the powder from a funnel and constitutes an indication of the flowability of a powder. By way of indication, a plugging powder having a difficult flow has a talus angle of greater than 55°.

The composition thus prepared was packaged in powder form in a water-soluble PVA-based sachet. The packaging article thus obtained can subsequently be employed as a care composition: it is placed in the hollow of the hand, water is added thereto, in order to dissolve it and to form a cream, then application to the hair, preferably moistened beforehand, is carried out.

The composition is distributed easily over the hair, makes the hair supple and makes it easier to disentangle.

### Example 2

The compositions A (according to the invention) and B (comparative) were prepared from the ingredients indicated in Table 2 below:

TABLE 2

| | Composition A | Composition B |
|---|---|---|
| | (invention) | (comparative) |
| Soybean oil | 1.0 | 1.0 |
| Isopropyl myristate | 2.0 | 2.0 |
| Sunflower oil | 0.5 | 0.5 |
| Coco-caprylate/caprate | 0.5 | 0.5 |
| Amodimethicone | 0.7 | 0.7 |
| Behentrimonium chloride | 2.4 | 2.4 |
| Cocamidopropylbetaine | 0.2 | — |
| Isopropyl alcohol | 0.5 | 0.5 |
| Hydroxypropyl starch phosphate | 2.2 | 2.2 |
| Maize starch (Zea mays (corn) | q.s. for 100 | q.s. for 100 |
| starch) | | |
| Water | 7.5 | 7.5 |
| Magnesium carbonate | 7 | 7 |
| Sodium bicarbonate | 3.75 | 3.75 |
| Citric acid | 1.25 | 1.25 |

The powder ingredients (maize starch, starch phosphate, citric acid, guar) were mixed with stirring in a vessel. The liquid ingredients (except for the fragrance) are heated to 40 °C. These ingredients are added as binder to the vessel by spraying and mixed. At the end, the fragrance is added by spraying and mixing is carried out. The combined mixture is subsequently dried in an oven at 45 °C for 20 hours. After cooling to ambient temperature, the mixture is ground using a mill in order to obtain a powder.

Each of the compositions A and B was applied directly to locks of moderately sensitized hair (AS20 locks) weighing 2.7 g and moistened. The amount deposited was 0.35 g per lock. The lock was kneaded gently between the fingers over its entire length.

After a leave-in time of 2 minutes, the locks are rinsed.

A panel of people evaluated the breaking up and the cosmetic properties conferred by the compositions A and B.

The composition A according to the invention broke up better than the comparative composition B. No lumps were observed on the hair with the composition A.

The cosmetic properties in terms of disentangling, of smoothness and of suppleness were evaluated sensorially, under blind conditions, on wet hair.

In order to evaluate the disentangling, the expert evaluates the ease of passage of a fine comb through the hair, on sliding it from the root to the end.

The evaluation of the smoothness characteristic is tactile. The expert grasps the lock between the thumb and the index finger and slides his/her fingers along the lock from the upper part as far as the ends; he/she evaluates whether the hairs exhibit bumps, whether they are not caught by the fingers.

Each of the 6 experts identified the lock which was easiest to disentangle, had the smoothest feel and was most supple.

In order to evaluate the disentangling, the expert evaluates the ease of passage of a fine comb through the hair, on sliding it from the root to the end.

The evaluation of the smoothness characteristic is tactile. The expert grasps the lock between the thumb and the index finger and slides his/her fingers along the lock from the upper part as far as the ends; he/she evaluates whether the hairs exhibit bumps, whether they are not caught by the fingers.

The evaluation of the suppleness is tactile. The expert takes handfuls of hair and tries to fold it.

The more easily the hair can be folded and bent, the more malleable it is, the better the suppleness.

The locks treated with the composition A according to the invention exhibit an easier disentangling, a smoother feel and an improved suppleness relative to the locks treated with the comparative composition B.

1. Solid composition comprising:
i) one or more cationic surfactants,
ii) one or more starches,
iii) one or more amphoteric surfactants in a total content ranging from 0.01% to 10% by weight relative to the total weight of the composition,
iv) one or more fatty substances.

2. Solid composition according to claim 1, characterized in that the cationic surfactant(s) i) is or are chosen from:
the quaternary ammonium salts of formula (Ia): 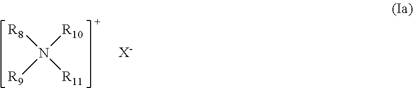
in which:
the groups R8 to R11, which may be identical or different, represent a linear or branched aliphatic group including from 1 to 30 carbon atoms, or an aromatic group such as aryl or alkylaryl, at least one of the groups R8 to R11 including from 8 to 30 and preferably from 12 to 24 carbon atoms, it being possible for the aliphatic groups to include heteroatoms notably such as oxygen, nitrogen, sulfur and halogens; and
X— is an anion;
the quaternary ammonium salts of imidazoline of formula (IIa): 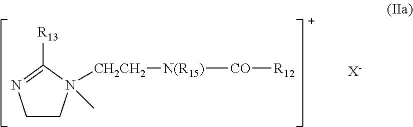
in which:
R12 represents an alkenyl or aryl group including from 8 to 30 carbon atoms,
R13 represents a hydrogen atom, a C₁-C₄ alkyl group or an alkenyl or alkyl group including from 8 to 30 carbon atoms,
R14 represents a C₁-C₄ alkyl group,
R15 represents a hydrogen atom or a C1-C4 alkyl group,
X— is an anion;
the quaternary di- or triammonium salts of formula (IIa): 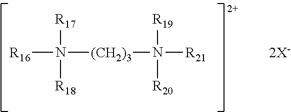
in which:
R16 denotes an alkyl group including from 16 to 30 carbon atoms, which is optionally hydroxylated and/or optionally interrupted with one or more oxygen atoms,
R17 denotes hydrogen, an alkyl group including from 1 to 4 carbon atoms or a group —(CH2)3-N+(R16a)(R17a)(R18a), R16a, R17a and R18a, which may be identical or different, denoting hydrogen or an alkyl group including from 1 to 4 carbon atoms,
R18, R19, R20 and R21, which may be identical or different, denote hydrogen or an alkyl group including from 1 to 4 carbon atoms, and
X— is an anion;
quaternary ammonium salts containing one or more ester functions, of formula (Va) below: 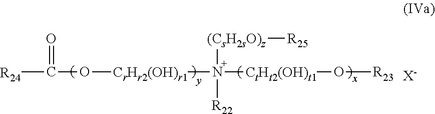
in which:
R22 is chosen from C1-C6 alkyl and C1-C6 hydroxyalkyl or dihydroxyalkyl groups,
R23 is chosen from the group R26-C(═O)—, linear or branched, saturated or unsaturated C1-C22 hydrocarbon-based groups R27, and a hydrogen atom,
R25 is chosen from the group R28-C(═O)—, linear or branched, saturated or unsaturated C1-C6 hydrocarbon-based groups R29, and a hydrogen atom,
R24, R26 and R28, which may be identical or different, are chosen from linear or branched, saturated or unsaturated C7-C21 hydrocarbon-based groups,
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10,
X— is an anion,
it being understood that r2+r1=2r and t1+t2=2t, and that the sum x+y+z ranges from 1 to 15,
with the proviso that when x=0 then R23 denotes R27 and that when z=0 then R25 denotes R29,
amidoamines comprising at least one C6-C30 hydrocarbon-based chain, preferably corresponding to the following formula (Va):
RCONHR″N(R′)₂ (Va)
in which:
R represents a linear or branched, saturated or unsaturated and substituted or unsubstituted monovalent hydrocarbon-based radical having from 5 to 29 carbon atoms, preferably from 7 to 23 carbon atoms, and in particular a linear or branched C5-C29, preferably C7-C23, alkyl radical or a linear or branched C5-C29, preferably C7-C23, alkenyl radical;
R″ represents a divalent hydrocarbon-based radical having less than 6 carbon atoms, preferably from 2 to 4 carbon atoms, better still 3 carbon atoms; and
R′, which may be identical or different, represent a linear or branched, saturated or unsaturated and substituted or unsubstituted monovalent hydrocarbon-based radical having less than 6 carbon atoms, preferably from 1 to 4 carbon atoms, preferably a methyl radical.

3. Solid composition according to claim 1, characterized in that the cationic surfactant(s) i) is or are chosen from cetyltrimethylammonium, behenyltrimethylammonium, dipalmitoylethylhydroxyethylmethylammonium salts and mixtures thereof; and more particularly from behenyltrimethylammonium chloride or methosulfate, cetyltrimethylammonium chloride or methosulfate, dipalmitoylethylhydroxyethylmethylammonium chloride or methosulfate, oleamidopropyldimethylamine, stearamidopropyldimethylamine, behenamidopropyldimethylamine, brassicamidopropyldimethylamine and mixtures thereof.

4. Solid composition according to claim 1, characterized in that the total content of the cationic surfactant(s) i) ranges from 0.01% to 15% by weight, preferentially from 0.1% to 10% by weight, better still from 0.5% to 8% by weight and even better still from 1% to 5% by weight, relative to the total weight of the composition.

5. Solid composition according to claim 1, characterized in that the starch(es) ii) is or are chosen from maize starches, rice starches, potato starches and modified starches, in particular phosphate starches, such as distarch phosphates, and mixtures thereof.

6. Solid composition according to claim 1, characterized in that it comprises at least two starches, preferably at least one unmodified starch, such as a maize starch, a rice starch or a potato starch, and at least one modified starch, more particularly a phosphate starch.

7. Solid composition according to claim 1, characterized in that the total content of the starch(es) ii) ranges from 10% to 90% by weight, preferentially from 20% to 85% by weight, better still from 30% to 80% by weight and even better still from 40% to 75% by weight, relative to the total weight of the composition.

8. Solid composition according to claim 1, characterized in that the amphoteric surfactant(s) iii) is or are chosen from (C₈-C₂₀)alkylbetaines, (C₈-C₂₀)alkylamido(C₃-C₈)alkylbetaines and mixtures thereof.

9. Solid composition according to claim 1, characterized in that it comprises a total content of amphoteric surfactant(s) iii) ranging from 0.05% to 5% by weight, and even better still from 0.1% to 1% by weight, relative to the total weight of the composition.

10. Solid composition according to claim 1, characterized in that the fatty substance(s) iv) is or are chosen from vegetable oils, liquid fatty esters of fatty acid and/or of fatty alcohol, and mixtures thereof, better still from sunflower, maize, soybean, marrow, grapeseed, sesame, hazelnut, apricot, macadamia, arara, castor, avocado oils, isopropyl myristate, coco-caprylate/caprate, amino silicones, such as amodimethicones, and bis-amino silicones, and mixtures thereof.

11. Solid composition according to claim 1, characterized in that the total content of the fatty substance(s) iv) varies from 0.1% to 30% by weight, preferably from 0.5% to 20% by weight, preferentially from 1% to 15% by weight, better still from 2% to 10% by weight, and more preferentially from 3% to 8% by weight, relative to the total weight of the composition.

12. Solid composition according to claim 1, characterized in that the fatty substances are liquid and their total content varies from 0.1% to 30% by weight, preferentially from 0.5% to 20% by weight, more preferentially from 1% to 15% by weight, better still from 2% to 10% by weight, and more preferentially still from 3% to 9% by weight, relative to the total weight of the composition.

13. Solid composition according to claim 1, characterized in that it also comprises one or more cationic polymers, preferably chosen from cationic polysaccharides and mixtures thereof.

14. Solid composition according to claim 1, characterized in that it also comprises one or more polyols, preferably chosen from those of following formula (XII): 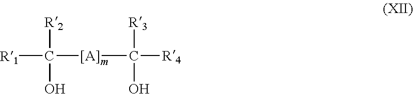
in which formula (XII):
R′₁, R′₂, R′₃ and R′₄, which may be identical or different, denote, independently of one another, a hydrogen atom, a linear or branched C₁ to C₆ alkyl radical or a C₁ to C₆ mono- or polyhydroxyalkyl radical,
A denotes a saturated or unsaturated, linear or branched, alkyl radical containing from 1 to 18 carbon atoms, this radical comprising from 0 to 9 oxygen atoms but no hydroxyl group, and
m denotes 0 or 1.

15. Solid composition according to claim 1, characterized in that it also comprises one or more C₁₋₆ carboxylic acid(s), preferably corresponding to the following formula (XIII): 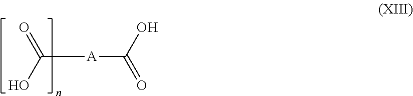
in which:
A represents a monovalent group when n has the value 0 or a polyvalent group when n is greater than or equal to 1: A represents a saturated or unsaturated, cyclic or non-cyclic, aromatic or non-aromatic, hydrocarbon-based group comprising from 1 to 6 carbon atoms, optionally interrupted by one or more heteroatoms and/or substituted by one or more hydroxyl and/or amino groups; preferably, A represents a monovalent C1-C6 alkyl or phenyl group or a polyvalent C1-C6 alkylene or phenylene group optionally substituted by one or more hydroxyl groups;
n represents an integer ranging from 0 to 10, preferably from 0 to 5, better still from 0 to 2;
better still chosen from salicylic acid, citric acid, glutaric acid and lactic acid.

16. Cosmetic process for treating keratin fibres, in particular human keratin fibres such as the hair, comprising the application to said keratin fibres of a solid composition as defined in claim 1, the solid composition being applied directly to said keratin fibres or after having been moistened beforehand with water.

17. Packaging article comprising:
an envelope defining at least one cavity, the envelope comprising one or more water-soluble and/or liposoluble compounds;
a solid composition comprising i) one or more cationic surfactants as defined in claim 1, ii) one or more starches, iii) one or more amphoteric surfactants as defined in any of claims 1 and 8 to 9, and iv) one or more fatty substances;
it being understood that the solid composition is in one of the cavities defined by the envelope.

18. Cosmetic process for treating keratin fibres, in particular human keratin fibres such as the hair, comprising a step of using a packaging article as defined in claim 17; preferably, said cosmetic treatment process comprises the following steps:
i) mixing the packaging article in a composition that is capable of dissolving, totally or partially, the envelope of said packaging article,
ii) applying the composition obtained in step i) to the keratin fibres,
iii) optionally leaving to stand,
iv) rinsing said keratin fibres, and
v) optionally drying said keratin fibres.

19. Use of a solid composition as defined in claim 1, for caring for keratin fibres, in particular human keratin fibres such as the hair.
